SEQUENCE LISTING (10 sequences)

SEQ ID NO: 1 - DNA, 40060 bp, Homo sapiens
gccaggctccacgtcaaatccagtttgaaacacagaccctaggaccacgcaggaggtggtgggctcgcaggaaggttcctctcccagtggccatgggtagcaacagtgggcaggctggccgccacatctataaatccctagctgatgatggcccctttgactctgtggagccgcctaaaagacccaccagcagactcatcatgcacagcatggccatgttcggaagagagttctgctacgcggtggaggcagcgtatgtgaccccagtcctgctcagcgtaggtctgcccagcagcctgtacagcattgtgtggttcctcagccccatcctgggattcctgctgcagcccgtggtcggatcggccagcgaccactgccggtccaggtggggccgccggagaccctacatcctcaccctgggagtcatgatgctcgtgggcatggctctgtacctcaatggggctactgttgtagcaggtaagtgggctgcacaaggtgggcagacagggatatgtgtcttagtgtgtacctctgcaggaggaggatgttcatgtgtttgacctttcctagaaatttgttagaattgatctctcctaggttacaatgaacttcccctttgacaaatttggcccagattctgtggttggggagcctctacttctagaaatatcaaaataaatatgtttcattgttgtgaagatttaaaaataggattaaatttgtatctattttattaaatttgtatctattttatcgaattgaatatttttacatatttatcaattgagttactttttgagcccattatgctctatttaatactcttatcaaattgactggctgccaaatagctccataaatatttacaataagtcacctgtgtatccaaataagttaattctgttcatagttttatgtagagcgttttaataggcgtagggcataagcccatcagctcaagaggacctttctggcattgcaaattctggacaaaagcaaacctgagggtaggcattttttctagccattaatcagctaccaattaatcagtaattgatggtagtacaataaagttgcattgcaaaaaggatcgctaaaaatagcacacatttgtagatcattaactctcaagctagcagaatcggtgattatgaggcaggataatcaattatcatttgtacatatttgaaagaaagcgtaaatgtacaaatgtaggatatttccaggagaatgaacaaaaagctgataagcattgttgactctaaggaggattactgaaggacaggaggtgagaagtggcgggtagacttattgttcatttaattttatactgcaatttttaaaacgtgtatataatctatgctattcttaagtttaaaaagcaaatggaaacaagagaaacaacaatgtatcttttataggagattgggtaaacgattacagcgtatccgtagaatgaaaatccagctgttaaaaattgtgaggagctgaaattatatgaagaaagttggttactgcatcagtgctgttcaaagatgtggcctctgacctgcccctcagcatcagcagagaccgttagagatgcagtttctcgggcccccttcaggcccaccaattgaatcagagtcttgggaaatggggccagcaatctgtgttttaacaagacatacaagcgatagttcaatatgccagactttgagaagcactgcgtttgactaccattgtacacgtgtgtctgtatgtgcagaagtgcgtaaaaggaggccttaaagcctgacctttgctggggctgggattggaagcatctccctcagcttttttaatagaattggaattgcttgcgataagcatgcatacttttttatttaaggagatattttacataccagaaatttcactaattaaaagtgtacaattcagtgcctttagtatgttgtgcaaccattaccactctctaattctacaacattttcatcacccccaaaataaacccattaccatcaatagtcacgtccacttttcccctatgcaccagccctaagcaaccactaatttactttgtgtctccatagatttgtgttttctggacattcgcttgcataatttttaaaacctgaaaaaaaatgtaaagctatttttatttggaaaaaagcaaaataaaagcaaggaagatgattttatggcaagaagtttaggtggaaattacaaaacgcggatgattctaaaacaggatttaggagaccaatgtttatttttcttttagctttgattgctaacccaaggaggaagctggtttgggccataagtgtcaccatgataggtgtcgttctctttgattttgctgccgacttcattgatgggcccatcaaagcctacttatttgatgtctgctcccatcaggacaaggagaagggcctccactaccatgccctcttcacaggtagggaatattccagaaagtctctccttcagctccccagacaatgaggcacttccactaaagccatccagtgtctctacacgtggagttttttgaatgaacgggtcagctgatgggctgctcccgtcatgcgggcactgtctccccgtgcatttctctgaacagtcagcttgtgactagtgctgcagtttctgcaaaggaagtttacagaagagctttcttaagaatgctttgtggagacaattacaactgaaatggaaagaagccttgtggattgtacttttgcattagagcagaggatctcaagcatcagcaatgtagcatccacattccttagaaggtcattgaaaatacagttgcctgggctccttctccagttattctgattcagtgagtctaaggcggggcccagacatttgtattttatttaataaagaccccagatgattttgtggcaggtttaaaaaacaaaaagagaaaacaaaccaagcaaacaaaaaactcaacgtcttagaaatttcttagttatgtaaaaggttggcatttcctcaaccttgtggtttgaaaaagttcatgccaggaggaaaatgggagctctcttgatctcttgggactgttgccagcctactttggttactgttggtcaattgtttagagcaatgctactcagagcaccagttaggactatgctagaactaggcattgaaatataaggcgacaggaggatcctggcccaagaggatttttatatctaagagggggactctatcagcccaccttatttgattgtctttgtagcccagccaatgcctttatttatgttttgatgtattctctctccttccatggccaggtaggtccatgaaaagcaagatttcaagttttgctcatagtttctgtctctagagctaagactgctgcctgcccatggaagccactggagccaatatttgtcatttgagcaaacatttgaatagttgaaaacatgaaggagtgaggccgttggagcacagttggtagatcctcctctgtgtaaggaaggcagaggggcaggagtgcagccccgtctgtcactggcccctgcccactatgtctccaggtgtttaaaggcacctaagacatggccagggcttgagacttcccctgcctcacctttgctcttggcttcccttcccactgcactttccacgtgtgtctcctcactcgggtgctcccaccccgctggcgtccttctgtctctggggtcagcccagcccatcatacctgccccagggtttggcatgagctcttgccctgcctgggacacccttccccatataatcccatagtcatgcctttattccactcagatttctcatcagatatctctcatcagtgaggtctttcttggctaccgtacctggcatagtatgaccactcctctttctcctcatccttattgggtttgatttttctttctagtgcttatcaataccagactaactgactggctatttatttatctatcatctgtctctctctctccttctcttttttctttctggttgtctctcccatttagaacatcaatcccagctgagcagacctgttgttttgttctctgatgaatcttcagttcctggaagtgtctgctgcatagtaggtgctcagtaaatactgaacaaacatttctcaaataccccatgtatgataataaccatgtattaggtattttctcttacattatctcgatcctgacaatggccatgtggaagtggtgtgattcccatttttcagatgtggaagctgagccttgaaagcagtggtgtgcttacagctggctcatgctggcttcttgagaatcaattgtcaaattttaggaattcagtgaacttgttgacatcatattgctggcttgaaactggccatgatggcagtattaaaactctagaaattggcaaagctataaatcatggtttttttttttttcttggaaagccagttgttaaatatttacctgaagtccactgattggagtaggaggggggttcggtgattaagtcacttgcccatagtccatagccggaaagtctcattgaatagattccaacttcattttcctaacttgaatctgacaattccttctgttttattataatttgttgaaattaattacaaggtcatgggagactttctggcttttgaactcgatattaagagtttattgatctacaggacattggacaatgtggccaccacttcccagcttacaattgtattagatgtggctaaaagacccatgctttgctgccttgacattaaaagacaaaactctgtgttagggaaacttttccaaggccaatacatgatagtattgtaaaccagaattaaaattctaacccccagctaactgaaggacccttcctctcggccaaggtcatcctaaagtaaatctgaaatactagtttaggccatgatgggaatgggtggttggacatgcctcattatacctctgtccctttggaattcaagcacaattgatcagcattaacattaaagcagagactttaaaactgacaaaacagactgtttgtagcaattaggtattaacatgataacatagcaggccctgaaagaaatagaagtatttcaccccaatatacatttatttgatatattttgaaatggccctgaaaagctgtctcttatggggaaaatctaccttctgtactcctcactcttccctttccagttattttcctgatttaggatagaattaactaaagtatctggcacctttttaagtctgataggaaacgtttacagtctattctctctgaagcctgctacatggaggcttcatctgtataataagaaccttggttgccacaaccccttattttaacccagacgctctcttctattgcttccaagtctatagataactctttcaaccaattgccaatcaggaaatctttgaatcttcctatgacctgggagccccactttgatttgtcctgcctttccagaccaaaccaatgtacatcttacatgtattgattgatgtctgatgtctccctaaaatgtataaaaccaagctgtagccctactgccttgggcacatgtttcaggatctcctagagctgcgtcacaggccatggtcttcatatttggctcagaatatatctcttcaaatattttacagagtttgactcttttcattgacagtatttttatgagtttcagacaatatatcccttttgagattttctgttgatattagtatatggtatggtgccagcaaacatcaataattttaatattaccttcattcaggctcttagtacatctcatctgagctttagtactgttgatggaaaaaaattaaaatatttgaagaagtttattctgagccaaatatgaatgactatggcttggaacacaacctcaagaggtcctcagaacagttgcccaagttgtttgagttatagcttggttttatatattttagggagctagaagttacaggcaaagatataaatcaataaatgtaaggaatacattggttcggcccagaaagttggggcaccttgaagaggggggctcacaggttataggtagagtcaaaactttttcaattggcaattgattcagagttaagctttgcctaaagagttgaagtcaacagaaagaaatgcttgagttaaggaggattgtggaagccaaggttcttgttatgtagatgaggcctccagatagcaagtttcagagagaatagatggtaaatgtctctttttgagtcttagaaggtgtcaggctctctaaaaaaaaactggtaagggaaggaggttctctacaggaaaaaaaatcccccacaaaagatggctttgcagggccattccaagatatatcaaagaaatatattttgggtaaaatgcattaattttcttcagggtctgttatctgtcatgtgatggtataccagagtcaggttggaatttggcatcctattgctatgaagagtgttttgtatggcagtcttaagatctctattttaatgttaatgctggttaattctgtctaaactccaaagggaagggttataacagggtatgtccaacacctcccttctcatcatggcctgaactaggttttcaggtttctttgggatcctcttggccaagaagggtgtccactcagtcagttggaggccttagaattttattattggtttgcagtactcatcctttaactgttctccagtctccagtttcttcccactccaattgagccaactgatagcttccagattcatatttttaaaagccagccgtgatcaggacactttcctgcttgatgagcttcagtggctccctatgtgcctcatgggtaaataatattttcctagacagaggttcaaagctgccacaatctgttcccatcctactctccaagctttacttcccattactccttccacattctctgtgctctgaataatttttcatcatgtttccaggagcagcgggcaggtctgttttattccctgcatccctcactcctctaccactgccttgcatattgtaggtgttcaaaataaataccaatgggacaaatgaaggagccagcccttccttcttcaggttctgggtctttgctcacatcgaaagtttctttctccttttctgcctgtggagatcaaagctgcttggaaatgctcactcattttctaggaacaagggccctcattctcttcgaaactgcaggtatacttaatttctaacttggtcggggactagatccacaaaataatcaaagcacaaaagtttattgagtagatgaacatgaaggtgccagtgaggtagataactccatcaggaagagaccattgaggctgcagttgatcacacatacatcctgaattttcatctcctcctggggcctttatggtctgtttgggctgccatgccagaatgtcactgactaggtaaacagaaattcattttcccacagttctggagtatggaaatccaagatcaagaagctgtcagggttggtttctggtgaggcctctcttcctggctggcagacagccgccttcttgctgtgtcctcatgtggcctttcttctgtgcacctgcaccctcactgtctcttccttttcttacatgaacaccagtcctattggatttgaattagggctccacacttacagtctcatttaaccttaattacctctttaaaggccttgtcgacaaatacagtcccattgagggttagagtatcaacacaggaatcgggagtacagggacacaattgagcccataattgagccctttcacttttacccttgttgccgctgagtccagctcctgaagcttggatgggatgtggatggagcacacatgcttttctctctcttccctcctccccaatcccttttgttttccttctttgcataatgaagatattctagaattttcaaataaacaagaaaaacacaaaaggtctttttgtgttcataagagcagttatggaactggagtattgtgttccaatctagctgttctcaggttctcatgcagaatgctccctttaagatcttctaaaatccctccacccctgtcctgcagggaggggaaaaaaatctcccccacccccctcacccctccaacctccaccccatccttggagggaggggcagttggtggcaaagggaaagttggctcacaccctgaggaaaacattttaccacctcccagcttagataaacagtccagccctaaagggctactttcttatgtctctttcttggcctttgccattttccaccttccatgttagttatttgaatacatttctaatcatactcattacaccataaacttcttgagggaagggggctgtccagaggtaacatacgtgtgaatgagtgagaggataagggatgtgtgaatgagagacaatgttatatgcactttaatgatctttttgggggactggtactgccacccaaaaatccaggttgatttccttcagcccttccccgtgttctcggccccttccctatcagcacttggtatatgcttagggaatagagtgattaggaaggggttgtttgcaagtcactaacgcccacaaaacagtggcttcaaggcagatgtttatttttcttatgtaacaaaaagtctgggagtacgtgtctgctggctttcagtgagctgctagatgtcagagccaacaatttaatgtttccttggcagctttaaggatgtattgcctcctggatgcaagacaattgctacgggtccatagctcttgtttgtttcaagacaagaggaggggagaggagggtcagggagaaggaagggggaatggggaaagagcctacattcactctttaaatgaagaaagcaaatctgccctagaaacctcccctctcccagagctctgcttgtttctcattaggcagaattgggttatgtgagcattcagtggcctaaggaggttgaaaagtgagggttttgctgggagttggtcatgtatctccctaaaccacattggggttttattagcaaaaaggaaggagcaattacaattgaatagacaaagctggtagctatcacataaaatgcaaacacagttgaatgttagcttaataagaataattaaagaccaagactattataagaaaatgaattagagcttttaaagtcccatatatgagtaaataattaaaggataattatattttggagtgatatacatgcctgctcgcaattttacctgatctgcacatttgttcactctctcccctgtattgtaatttacaaggtatttatctacccctcctgcaagattcgtaaactctcccatgagggtttacctgcaaattccccatggcttctaattcattgccttgctgttagcaggcacttaaacatgttagttgtgtaaaaggagacccgagggttacataaccctgtttataattcatggttttaaatgacacgacaaattgcatcaattagacaaatatgctcatttaattactttgtctcatgaccagcactgaaccccgaacctcagccgactgtctcccgtgcatgtctttgctcatatggaagcagcagaggttgtgtggatggctcagagcaatttctttccccaactagaaaaacaacttaaaacatcttcctcttttaggacagtgcagtggagtctctcactttgtttaaggcttagattctaaagtccccaactcagtgacaatgacaatcgacattattcgtaaatatctctcagattactcataaaatacaatatgctttgttttgtgtgtacaaacctgcacatatatatatgcagatttgtctctcctttaatgtttactcctgaaattaatagaattacccatactatttaaaatgttactcttgtctttcttgcaatctctctctttctccctgtgttcaaagtgtgaatagttgaattctggtgagttaaatgtgggatatgtgatcccactgtggtatcctcactctgaatgaaaaactgcattaaaagatgagtgaaaataacaagcagggccttgtccctctgccctgtctttgcatacatgccaaactttttgaatttacatgcccttgacaaagagataactacagcattccacttgaagcaaacgttttccggcagaggcatgttcagtgtcagggcccattttgatttttcatcaacagcaaaatattattcacccaaggaatccaatcatcccagtaaactgaaattctttttttttttttgtttcactgctgtggttctgttccagcatttccaaatgcttcataaataagcatccaagggttcattaccaaaatatttcccagcatatttctgacatttttttgggattttctagagaaaagactgtactggctcctagggagacatttaattcctctagcttctatctttcatatttaaaggcaactctatcctcctccatccctggcatatgtggtagccatggtaggtttggtgggtccacccgcatcttctcaccacacagatccttcactcctgggtgcacctgaggctgtggaaccgagtgagctaacagagtggtactcaccagggaagactggaaatccttttgtgcccagggagtctaaccaagcgtatttctgctacttgaacccaagactttgctgttcttagcagtgggctttcttgcttggcgggaggctgggatcaggcatgacaggaaaggccagccctgttcagctcctggttcctcctattcactgcagagtagcctcagcatttctgagctttcttcattccttcacaggcttgcaaatctctgcaccatgttagttaataaccaccttgcaagggtcacaggcacagcacaatgaaccatctaaaggatttcatctaacccaaagagatgaaacgccaaatgtttatatttacttgatttccgctatcctcactacgcctgcaaacattttatttgtaatgactcagtgagaaacttgccccattacatccttggaagaagaatgggtaacttgtcagatatcaacttcttttaacttttgtcttttaatacttcaaagtatgcacaaaaaatgcgaacatgcaacaaatgctctaaatagcaaataattaagggctaagaaaagagtcttaaaaacgaaattgaaacaagagactctttgactaaaccttttacacagaggaaaggagtcacttcatgtttgagactgttttaaccttttctctttttcatctttagatgagtggttttgtttcatcaaacaaaacctttgaagttccacccatggtagactttcacatctcctcattcaggcagcttggaaaggatatggccagggcagaaaccaaggatacagggatttcagcttgtggataaaaagggtgatttatgcaatttagggtgattaagtctccaaattgccatctcttttccattcaaatgggatcaacacaactctcaaagaatctccaagtcagattagagaagtgggaatctgggggactttgtcctccctgtttcctctgctcgaaagtgctttcccaggtaccaattggcagttggcttttggcctccttgctttgttactctgatcgcaaacacacacccccagccctgactcccatcacagaggtacccatcattacttgacactgtattattttttcatttatttatttctgtaggtctgaatcaaacagattttattcaactttttagaatgagggaaacaaatgatataaaataagtcacaaaaatgctttcttatcctactacctcaaaccattttcaatgttttacaaaatgctcacctagcaaatacgaaaagtttcaacacattcttctcttgtaacttctgctgcaataaatgcgatattagcaaacatacatacttccatatcagtcctaaaagttggattactgaattattatactaatttgtatgatgttactcatatttttattcatacacttttaatgagatcattggcaatacgtatagtattttctttaacttcactttcacattaagccaatgtcttttatatagccatcaaaaagtaaattacacaggtttatagttcaatttaagctctagcgtcttagaatcagccactttctaacctttcatgatgccttcctgctttgcaaaaccatgatcagcatgaattgcaaacatacctccttcagtacaagcatttctcaggtctcctccattaaagtcatctgcaaacttcacaattactccataattaatttcaccatactttgtaaggggacttgcatggattttcaacgtgtctaatcttgctcgttcatttggcaaatcaatatgtatttttttctatctaatcttcctggatgcagcaaagcaagatcccgtgtatttggtctgttgtattttaactctgcgcagagtatcaaatccatccatttgattcagtaactccattaaagttctctgaatctctctgtcagctgaagtacccttggaaaaccaacaaccatcaatagcatctaattcatccataaaaatgatgcatggtggatggtccctggcataattaaacatttctctgatccaatgagccctttcaccaatgtgcttgtctacaatagaactagatacaacctttaagaaattgcagtcctggccaggtgtggtggctcacacctgtaatcccaacactgtgggaggccaaggcaggtggacctgggcaccatggtgaaaccccatctctacagaagatacaaaaattagctgggtaactgggtgtggtagctctcacctgtaatcccagtactttgggaggccaaggtgggtggattacctgaggtcgggagttcgagaccagcctggccaatgtggtgaaatctcgtctgtactaaaaacacaaaaattagccaggcatgggggtgcatgcctgtaatcccagctactcagaaggctgaggcaggagaatcgcttgaacccagaaggtggaggttacagtgaaccaagatcacgccactgcactccatcctgggcaacagatgagactccgtctcaaaataaaaaataaaaaataattaattaactaattaaaaattagttgggcatggtggtgcacacctgtggtctgagccactccggaggctgaggtggaaggatcacctgagcctgggagatggaggttgcagtgagccaagaccacagtactgcactccagcctggacaatagagtgagaccctgtctccaaaaaacaaaaaaaaaggaaaaaagttgcagtccagctggccagcaatagcttgtgtgccaagagtgtttttcctgtttatggtggtccacataacaaacagccttttggaggtattattcctacaagttggaataactgggtttgtaagtggtaacccctcaattcccacatctattctgatagccctccaatctcagaataagaaacatcccaggatcctcatgagacatgttataaatcagtggctccacctctcttggtaaatatctcatgatagttagtgtagccctatccaaagcaactcttgttcctggcttcagctacttttgtgaagctgtcgacgacaactcccaacacatcttggtccatctgcagctttaacaatgaatttttcttcagttaattgtttaagcatgttactcacaatctgcccaacactttttagggccttcagatcattttcagacttttcatacggtttggtaaattctttgaattgttcccttagctccttaagatggtgcccatctccatgcactctggcagcttcttgctgtagtcctgaaatatcttatctctagggtccaccatgatgagaagtgctatttgtttattgtttattgtctgtctctctaaccatcacataaactcctcaagtacaagaacattgtgtttcctgttcattgaccaccactatcttgaatagtgcttggaatatggaaggtgctcaaaaaatattggtgagatggatattcagttttaacatcttaattttattaatgattaagctgtgttgttgagaggtaaagtggctaacccaaggtcacacagctggttagtggtagactaggggtaaaaccaaccaaccaaccaaccaaccaaccaacaagccagtcagccaaccagctaaccaaagatgtggttcatgagttagtctggattcctctaaaagttgaaccccagaggtttgtatacagatagtttatttgggaagggattctagagagcaagagtaaggaaatggtggacaggaacaggccaaacaataagcattacagagctggctctgaggaggcttaggaagggcaccaaagaactctccatgtaggggatgaatgagggaagattaatccagtctgtactgactgactaagggtggtaccatgggttttgtaagccctgaaccccagctgtttctgtcttccctcagtttaggcaccatccagatgcacctgtccagtgcaaagctggccggagctggcttagatttggtcactgcagcagctgctggagcaagggaggcaggtggggaccgagaaggtttgcagtggctcacaggggctgtctaactcagctgatgacttctagtctgcagctttttcattctagcatttgtgccctgtgctccctggtcaccactaggaggagttaaagcattttgcaaaacgcttcacggtgtgctgagccattacaacttagaactggaaatattagttctgtaaactttaagaaagctcaagacagccataaatggctccagaacgaggccaattaatgcctgacctcctgcacagccttttcttcctgggattcttactgcctccatggggtctgaccaggaaatgaggcacctatcttcttgctcttggatccctcaagccttccaggggaggctctgctcctctcttcccaccctccaactctagctaggcccatcaagcaggcagcaagaccatcgccgcctaagggatttaccaatatgtgtgcacccatcatgaatgctctgtctagcctgggaagcctggttcttaaagcacattttctaatgaatctactctcgatgccttcgcagcctaagggctttcggaactctgaagcaagacaaaccattcttatttctctgctctctactgttacatactgtcctcaaagcaacgaggcatcctggcccagcttaaatgtgaaagaagggcccaatgagagaaagggatgccaggattagaggtaggtggatggaaacagtgagcaatattttaaatatattatctcagcatctttgaagagaaaagttgctttgtaaacctaaagaaatatcagatcagcttgtctggtcaagctggtctttgaccagagcatatgtatcattttttttttattttaatttaaaattttttttttcttttcattgggaacacaacacatgtatcattttgactgctttttctgtactcagcctccaagagggcagaggtcatgtctgtcttgctcattgatgtatctcaagtgtttagcacagttcctggtgaagataggtgctctaacaatttctattgataaacaaatgggtcaaggacaccacacacacacacacacacacacacacacacagagagagagagagagagagagtagctggtacttatgttaactgtccaggatttagtatgtacctggtgggaataagtaggaagcatgcacctaatctgacactttcattgctatttttttcaacatgtagaaaagaatcagaacgactttctcatgaaccccatgggaagatgggagttctttagtctgtcctccttcagaggtaaagtagcaaacagcacatagctagctctcatgacttgagctgtgtcagccctggtgctgagcttgccaagccacacacctgatgcctgcaggcaattacaggcagatggtaggtgcagagtttgatttagatggactctcttgagtttcctttcatcctttagattccttggtactgacagtttaccaatttcccattttacctgtcatagttgtggactaaagacaattttttacatatttctatatatttggacttcttccagtatgggccactgttcaagttgcatcattagttatgaaatatgacatctgtcttctaaagccatttattctgctgagtttgacaacacagcctggcttggtgaggaaggggccagctggaccacttctggcccttctttgtaaaagcttgttatctaatgtggcagcagatgtcctccgaatgctctggcttatggcttgcccaatagcctggtgattgattccagtggcctcacagaggctgattttggcagcagaggtaaatacagtctggatgtaggtgtgggaggtggtaaaatggtaaaactatcagcctttatcatagcgcaattaggttttgttggggagattgtctttgaagataaaacccttggcttaagaagaagaagcaaaagaaaggcctttaatgaattcctgcagccctctactggccctcatcatatctgaggcagcaaatagataaagtttaaatatttttttccagtgtcttaatattctgttaggataattcccccagtggcccactttgctttcctggcatgtcgtgttgtactattctaacttgcagctacaactacttcaaaacagtagctttgggacaaataagacagttcttccctttttttttttttacccccttccctaagataagagagtgctaataattctctaagttatagattatgtcaaggttttaaattaacacattaataaaggaaccaatggggaagatataagaagagtttcccccaggttcttgcaggaaatgtgtgtgtgtgtgtgtgtgtgtgtgtgtgtgtgtgtgtgtgtgtgtctagcaaaataaaaaatgctagactaaatatgaacctggttaaccttctacaagacaaggggttataaaagtataacccctctgtaatacttttggagtaatttccctaccaggcaacaaaaaccctattcatatctgcaaactaccttaattttacaggactgcaaatcactgtgctttatcagttatgaattgtgggtcaaatatcctgtgacaacaaagtaccatttctcaagaaaggaagttagttagaaactgctgtcacctggccactcaaaagagtgatgcttggcccagcagcattggtgtcagtggtgagcttgaaagaaatgcagaatctcaggctccaccttagaccttcagaatcagaacttgcattttaacaaggtgcccacatgatttgcgtgcacattaaagtttaagaaggctgctctagaggaatattttaaaagtcaagcagttgcaatttttacactttccaagtttgagatatattttactttcttgatacttgctacatgccacaatagggtcaggggtaagcccaggtctcagccctcagaataatccattagttctaatcactgggcctaaaatgactaaatcctttcagcaagttccaggcaggctgccttcagccagttcagtgcagtagctatcttctgcttctaggggaggctttggaattagggttactataggaagtcagcagggagctcagttttgggtctggttctgccatggtatgtgctggggcttgacgtggattatttgcattaatcctcactaaattcagatgaggaggcagcaccacccttgtttctcagagaagtgcccaagagcatgtgactagaaagcacaaagataggaatggaaatcaggcacgattccaaatctcttcttccctctgtgtggtcttccatcacagagtcactaaagctcatagctttctgatctaactcttcaaatctccttttacatccaaaatttttgaattcattgatatattgaactcttataactttcattgctaatgaagagaaatcctgtcagatttactaataatcgacatcaccttgttactattaactagcaccaataatgactctttgaaactcctctgtactctgagtacccagctctgagttctagcttatcatttattactgttttgaaagtagcttctccggtggcgggcgcctgtagtcccagctacttgggaggctgaggcaggagaatggcgtgaacccgggaagcggagcttgcagtgagccgagattgcgccactgcagtccgcagtccggcctgggcgacagagcgagactccgtctcaaaaaaaaaaaaaaaaaaaaaaaagaaagtagcttctcccattatgtgcttctcccacttgaaaccaccttctggggaaaaaaatgggaaactgtgtcttttttatgtttatatccccagaagtttgtagagtcatttacatatatttagtgttcgatacatgtttattgaactaaagtaaaccagtagacatgcaactgcatcaggaaactccccaattgtaggactccattttccaaattagtctctaatacaaatccatcattatatgcatatccttatattttttccataaaataaaactaccattccattgcacagtggaggatttatttcacatatcaattctttcaaatgagcaggggtcttctgctctatattcttccaactgaggcaatgagtaatgctcagttgaacaaaaatttgaaagatagaggcataagtgataagaaattgaatggcaatgtgaaaacccttgatctgtttacaaatgatagtttcgaaaaaggaatgtgtctgtcatagggttgacttaggtcaggggttctaaactaggggccacttttcaacctccttccctcagagatatttggcaatatctagagacatttttggttgtcgtaatgggttggggagcagacttaatacattcttattggttgattgcttggcttgacgcatgacttctgataccttctgcctctctacaattgaatagtcattgtctaataataaccagaggcactacaaatcccagaacattcttctgaatcagtcgtagagtgtggctgtttcaggaccttctctttctctcaaatcctgggcacttcaccatcatctgtcctttatctgtttatgtatatgctcatcacatataactaggccataaatttcttgcttccagagactatatgtcttacttcactgtgccccattggccacagtttcttgtaattctgccttaaaacacttcagcattcaggacatgctaatgatgctttatttttggaatccaatgattagattgttggtcctccttaaataggctctatgttaaattgcaactaaccagaatatgaagtgaccaaaattgtctactctctcactaatttgaataaattaggattcatggtacttgacattcaggtttgtgttcttgaggtcagagtttatgtaactttagatgtttagggttagagaggaactcctttagttctattttcttccttgattctctagcttgtgaagatattgtccttatctgagaaataaaatagagacttcactcatacacacacacaaaaaaaatcaaaaacaaaaatgaaagctgatctgttcagctagataagttgtggttggtaggcatgatgggagtttgacgtttaaagaattttgacaggaggatcagtcctacataagtttgtgacatatgtagtggatgtgtccagcactcaactctttaattccctcgacatcccataatatgtccactgccccctccccaaccatgctatctccccacctttatcagaagtcactgatgtctgcaccagaaggaacatctgaggaacagttactactttggtgctgagtaatttcttgcccataccaggaacacgtgtattttaaaacagaaactcaaaaaaggaataactaataatgggattttaaatatcagataattaggattgaatagattttgaggagcctttcctcagtatccctcaagtgacacaatgtttaagcctgagaagataaaacaagcaccaaaaagacctcaggttggagtcctgggaaacgataataggatttttaaaaattcatgtttaaacttttgttctcattatttcctgttcccagctatcaactgggaacaagtaccttgagcttctaatagaggaagaaagacagcttttgagggtgtttcatctttgcaagcttacgtcatcccaggaagcttaataataagaaactgcaagtcatttaattttattgctgccagaaattggttttctttggggctttttctggtcagaaatttaagttcaaaagcagtctagagaatgcactttggaaaggtcacagtatctctgatggtagcaatacttagtaagcccttgctatgtgtctggccccatcctgaacactttctattttcaaatcatgaccctactagtaggttactcttttactattacctccatattctgggtgaggaagcccaagatcacagcaagtaaatgctagagccaaaacttgaacccacattgcctggctgtgtgtttttcagcctcttgctgtattgcttttccaattatatccaggttgcctctgctgtcttcagggagttttcccactgaaggggagtgtctatgcatgaggaaaatgaacgataatttgctaaatatgctaacatagcttttcttgctttccaggttttggaggtgccctgggttaccttttgggtgctatagactgggcccatctggagctgggaagactgttgggtacagaattccaggtcatgttcttcttctctgcattggtgctcactttgtgttttactgttcatctgtgcagtatctctgaagccccacttacagaggttgcaaagggcattcccccacagcaaacccctcaggaccctccattgtcatcagatggaatgtacgagtatggttctatcgagaaagttaaaaatggttacgtaaatccagagctggcaatgcagggagcaaaaaacaaaaatcatgctgaacaggtaaagatgcaacattttttctttacaagggaaaatattcttgctctttgttgttgttgttgtttttttaattgtttgttttgtctgtttgacgagaagagtttcatggggtctaactgaattcccaaatcccccgtagctatcatcccttggaattaaaattttatatctcaaagaaaaatgtctgcattttcctctggtcctcctgtctcttactcctctcaggtagcatccaaccagggaagatgaagccagttgagaaaggttaggcaacaagagaagcctaaaatcatccttaggccagaagaaatatccccttagattaggatcataaattagaaattttttcctccaaatggtagcatccttgggttggtgactgagttgggcatatcgaatttgcctgtcatgagttgattggatattttcgttcattttcagcgaactgcacagtgaattcatcagcttaaaatttgaaataaaactcaactaactgccagagaaatgagaccatgttcagatgaaatgatcagagaagagactagaattaagctgagcaatatagtagctaataatcccgtggggctatttaaattaaaataaaaataaaattagaacttcatttcttcagttgcattagctacatttcaaatgctcaaaggccccatagggcttctagctaaaatataggacgtgcagatgtaagacattcccatcattgtagaacgttctgttggacaacactgatgaaataattgcattttccttcaagctaaaattgaatgccaaaacctcagggatatagaacagaggatttcttgattgacaaaatgttgtagaagacttaagttcaaatcttggttcctctagctgctgacttgtgttattcattcagtgaatcacaaatagctacttgctatagggtaacctaaagattaagagcacagtgcttaaatcctggctgtaccacctctggattgcatgactttgggcatttacatttgtgttaaatgataaaataattccatctacctacaagggtttcgtgcaggttgcaggggctaattcaggtaaagttacttagagtgtttgcaaccacataataagctgttgggtttaggccttgttattttgaccctggcagaacactagagtgccttaagcacaaacagggaacacaaatctggactccaacaactttcagacaagctcagtgaatctcaccttcctgtgaggtcaacttcttttgttggcttggccagtctattttctaggattattaggatgttcaactaagataaagcatacaaagacactttttcaaacttcacataaatatatcaatacattataatcattattcatttaacaaacagcttggggtactgagtatgtgcaaagcactgtactagatatttggggaccataaaactgacaaatgtccagtctctatgcccaggaatttgcagcctaacacgtggcatatgccatttataccatgactcataatccatgatagaacatggtaaatgctataaaacaatgtatcagttcagatcttttaaaaaggcaccaagataggatttgacatgtaagagattcattggagaaaacagcatgaaagataaagtggagaaagcagaaataggaagggaaagtcttcttatgttgatgcaagtcaaacacctgtgaaaggagaaaggaaaggaaagaggatagggtaggaagcctctcagatgcagtgaagtttcaagaaagcttcactcaggccaaatgggagtcctccagccaaagttgctccttagaggagtcccaactcttgtaggaacaggctaacactaataccccccgtcttgttagtcactatctgggaacagccaggaaaggggtaactttggcaggaacaggtacaggaacagagttccttggcaggaacaggaacagtggatccagagaggcagcattcagcgctgtcgatcaactgtgttttctctggtaggtgacccaaacggcacatttccaaggtggccacaaatggcaaaatagcaattgcttttagttttaaagagggaaggattgtgcctaaataaatggagaaaagcgttatggaagagatcgcccctgagttgtgccttgaatgatagttaggatttgaatagagattttgggggaaaagtacttctgacagaagagcatcagcaaagccacagacataaaaaattttttttaaagcatgaagcatatttgtagaaagtataagtggtaccatagatcacagagtttgtaactttagaaataaagttaggaaagcaaattggagacacattttggaagcttttgaatgccagcataggaactaaaattttattaggtggatgatggaggtccatcaaaactagtggggcaaaggactccttgatgttagctttttttggttggttttttgtttttgatattaatccagctgctgactacagggaataaagaggtacccaggagggtttatttgggtgaataataagatgtcttggaatgagttgcactgtgtgcttggttcattcacttgtgcattcttttattcattcaatagctattaattacacctgccagacccagtgctgagcatcagaagactagtgaacaccacagcccttgttctaccccatggagttgtcacctagaaaagtgtgcctatgtgttattttgctttcttaatcaaagtttaattagatttacaaaaaagttgcagatagtagagagagttcctatgtatgcttcaccagcttttctcttatgttaacatcatagagaaccatgtacatttgttaaaattaagaaattaacactgcttcaatactgttaggtaaacagcagacttatttgaatttcaccagtttttctactaatgccctttttttaaaaaaaatgtatttccataggctattggggaacaggtggtgtttggttacatgagtaagttctttagtggtgatttgtgagactttggtgcacccatcacctgagcagtacacactgcacccaatttgtagtctttttcccctcatcccctttccacacttaccccctgagtccccaacgtctgttgtgtcattcttatgcctttgcatcctcatagcttagctcccacttatgagttagaacatacaatgtttggttttccattcctgagttacttcacttagaataatattcttcaatctcatctaggtcactgcaaatgccattaattcattccttttatggctgagtagtattccatcatatatataaaagatacctgcacacacgtttacagcagcacaattcgcaattgcaaaaacgtggaaccaacctaaatgcccatcaatcaacaagtgaataaaaaaactatgatatatatatatatatatatgtcagatgaatgtccataataaaaaaaatagatgttgacgtggatgcagtgaacaggggacacttctaccctgctggtgttgatataaacttgtacaaccactatggaaaacagtgtggagattcctgaaagaactgaaagtagagctacgatttgatccagcaatcctattcctgggtaatatcctttttctgatccaatctaggataccacattacatttagctaagcggtttacaaaatttaaaaacggtactttattttaatggtattttaaggaattttttttgctatgtatagtcagtagaaaacctcatttttttgtatacagttatgtatataaataaaagcaatacatcttagctgttagttataaaagtgtgtaagtagttttcccttgagatcaagtgcttcctccagtattgtttactctgttcatttgcttagcatttttccaaaataaatgcaaaagtaaacttcgacactgctcatgtttgaattggcaaaatggttgagtatgatctgctgaaacgaggcctcattctataaacatgactgccttcaagttgagttgccacagcgcatcaacctgcctctcgccatgccacttgataggagaatacagttgaagagagtaatctattcatctgattccaacagtagctcctattttaggatgagtgcagtgtttttcataactgtaagagtgcaacgaggaactaagaggtcattgggataaagccttcattttatctcaatcacacaagtagagaaaatttgagattttaggtctcctaatgcctggactcaaccccctttccctacaccaggctgcctggaggagaatacttgtactctttgactctccttcaccaagaagccagaactctaagattactagttggactggagccctctgtgcatttatgcaggacagccaaagaatcacggaatatctatatgataacattcatgcggtcattaaaaatgtatgtttccgaaggtttactaaaaatatatcaggtaaaatatacattacatgcacaaaaccatctttttttgggagaaaacaaagctgcataacatagaggaaaaaatggaaaggaaatatacaaaaaatgaaaagtctggatgataggctcacaggaagctttaattttctatttttaaatcctgcaatacatccaaaatttctataatagataaaaaaagggaaaaagtaatcaaataatgtgaaagtgaaaattaaaaaataatgtggtgatttggggaacaaaacaaggtgcggttttactaaataaaagtatatacgtgataatagataacaccctgatagattatctacctctttgatgtccccttcgatagactgggactccccctccaagagtcgcataggacaggggtcgtcccatccactcagaggctgctgtgaggatcacatgaaatcaactgagtggaagccttttgaaagtcatggaactcccacacgagtacaaaaataaatacaacagatacaacgaactttctagtaggggccacatgaaaaatgttgtagactctgaatgatcttgttatttggtttaaaaaacaaattgagaccagtgcagtggctcattcctgtaatcccagcaactcatgaggccaaggtgggaggatccattgaacccaagtgtttgatgctgcagtgagctgtgatcatgccgctgcacacttgtcaaagcaacagagagagaccccacctctaaaaacataaataaataagcaaataaaataagacaaaaagttaaagtaagaatatagcacagagtaatgcatcttactctcaaagccatggatttgcacttctcttccaaatgtgtgccctgggaggtctccagttaagcaaaggaagtctgcacttccccagggtcagggccaaggcttcagggctgtcattgcttgccaaagacattgctttccaaaataagggtggctgtgtaatttaagaacaaatttttcttttttaatggcaagagggctgaattatttcagaagtttaaaccagatctcataaaatctatctccaattcctccatgtcgtcagttgctccctttaacatttaaatagtctccttttgagggtatgtttgtgaagttttctttctccctgatgactctgtacctttttcgcttgtaaattcatcactacccagtctccagctgacagtggtgcaaaataaatgcagtaaaacaactcccaaggtttcagaatcattgaaaggtttttaccaagggaattgatgagtcaggaataatatcccagctgggtttttattctgagttaaaatatacaaaggggctttgaatatttacactctcagcttttacagaaatactatgcacattttgaaagctcatatttatttttagaagaaaggagaaattaggctaacaatgaaagcaggctgaaaattatgtataagtcagaagcatatattaatatttagaatgaggctgagtacttgctgaccagattcgctttctcagttttgcatttttggaaggagaggaaggaaagtgatgacaaaccatttagttggaggcatgaaaaccaggctgttgtttcttgggctattgttagctgcttctttctgtggttgaggaaaaagtagtaattctgcacttcattttggttataatcatttatttttacaggtaccatattgaacttgcagaaaaacagttattttccttcaatccagggagatgtctatagtgtttaacactgattatcagaaaatgagtatagttatggtcagtgggctagatactgcaagactgatgaattttaaaaaatcaacttacacaacaaaattaaaatgggcgaatataattttccacagcaaagaagaaaaaagcatgattatataaggttaagaagtataaaaaatatgctgttacagttaaaatagaaaatctcagtagactgtcttataaactagccagtactcaatgtcgtgattcagaataaataaaaatatgacaagaaagaccaattaccaaattgtgtctgatgtttatggtctcagtgtcttcagggaagctaatcaattaatattcccaaggtttctttcttctcttatatgacaagatgctaaaagctattatcattttttgtatttcaaaacagttgtatcaatagtttaaagtttctcattctctcaatgtttctgaaaccagaaaaactatagcttctagttttaacctaatttaaatctaaattaaggaatcggggcctatatattcattttttacatatatgtgtgtgggatgggccttcctaaagttaatggaaaaatagaattaaaagataaaaattaaaaagaataaactttatttcttaatgtaagctccattgagttcattgcttacttttgtaagcaatgataccagccgtttagcccatccctaaagaactgggggtcctggagatttagctatgtcaatgcagccttttttacaatattaactgaaaaaccatgggtgccctttatagattttttaagattaggaaacagaagtcagaaggagctaaatcaggactctgggattgatgcctaaagatattccatcaaaattatcacaacattgcccttgtttgatgataggaatgagcagaagcattgttatggtggacaaagacttcctggtgaagcttttgtgtgtttttctgctaaagctttggctaactttctcaaaacactctcatagtaagtagatattatcaatctttggccttccagaaagtcaacaagcaaaatgcctgagcatccccaaaaaaactgttgtcatggtcttttctcttgacctgtctgcttttgctttgactggaccaattctacctctcggtagccattgctctgattgtgctttgttttcaggatcatactggtaaggccatgtttcatctcctgttacaactttttgaagaaatgtttcaggatcttgatcctgcatgtttaaaattttcattgaaagctctgctcttgtctgcagttgatctgggcacaatacttttggtacccattgagtggaaggtttgctgaaccttaacttttccatcagaattgtgtgaactgaaccaactgagatgtctatggtgttgactattgtttctgccattaattgtcagtcctcttcaattagggcataaacaagattaacttttttatttgcaaactgatgtagatggtctgcccctgaggacttcatccttaacaccatctggttccttcctaaaatgagttatccatttgtaaactgctgatttctttgggacattgtcttcataaagttaaaaaaaaatcagtgtttcaccattcttctactgaagcttcaccataaatttgatgtttattctagcttcaattttagtagaattcatgttgctctgttaggggctcttttcaaactgatgtcttatccttcttagtgcctcaaactaaatcttcttcagacatgctataacaagttagtatgagtttatattgcttcaaaaaatttttggaatccatgcataattttttcataatatgcattttccattaacatattgaagaccgtgtgtgtgtgtgtgtgtttgtgtgtgtgtgtgtataaacatatatatttgagttctacattgttcttacttctaactcaatgtaaggctcattaacatgtttctgctatcattaggattcagagatataacctgggaggagacatctggctaagaacagggagctgactggctctctgtattaatgttctaatgaataaaaaattttcacaaacttagctgcttaaaacaacatcagtttattatctcacagttctgtagatcagaagcctgggtaagctgcactaggttttctgcacagggtcttacaaggttgaagtcaaagtgttagcagagctgtattctcatctggaagctcagtgggaagagcccgcctcaaagcacattcaggatgttggcagaatgcagttccatgtggttataggattgaggtccctgctttcttgtggactgttggcctggagttgctctcagcccctaaatgtcactctcaggtcctttcaagtggcttcctccaccttcaaagcagacatttcttgtgctttgagttgctctgactttcccttctactaccagctggagaaaattctgtacttttcaagggctcatggaatttggttaggatcaaccaggtaatctccctttcaattaactcaaagtgagcgattaataaccttaattattaatatatctgcaaaatccctttgccatgtaacagcacataatcacagggatgacatcaggggaccaagatcatgacagacatcttggatttgcctactatgctctcacagttaagagacccaagaattataatcatctaagagctggcttatgtctgtgagctttgtccttgtgcaacagttctgtcattcagacataggatgctcaatgtctcctacaccttggcctgaagctagcaccattctcattccctttgcccttgtgtccaaagcctcattctgggcctgaaactgccaagcagccactcgtgcttaacagaatacctcctgtgagtctactccatggaaacctagttccatgtccaggaaaagtgacactttgtgtgatggctgactgacaaatacataacatgtctgtgtgttctggctcccttagctggctgagtttctgcagtgaagatcacacctctgtttcatgttttatttcagactcgcagggcaatgacattaaagtcactgctgagagcactggtgaacatgcctcctcactaccgctacctttgcatcagccacctcattggatggacagccttcctgtccaacatgctgttcttcacagatttcatgggccaggtaatgaacgtgtctgtgcacacaatcactgttacacatcatttcctccattaacacctgttgaggttcttctgggctatcctctaagattggcacagtaagaatgtaggaaagggtaccatcaagaagctgacttacagacaggtgtagaaaaatgctactataccctggacaactagaggacactacggagcctgccagtatttaatcaaatacaacaaatagttgacagtggggtgttaaagtctcccattattaatgtgtgggagtctaagtctctttgtaggtcactcaggacttgctttatgaatctgggtgctcctgtattaggtgcataaatatttaggatagttagctcctcttgttgaattgatccctttaccattatgtaatggccttctttgtctcttttgatctttgttggtttaaagtctgttttatcagagactaggattgcaacccctgcctttttttgttttccattggcttggtagatcttcctccatccttttattttgagcctatgtgtgtctctgcacgtgagatgggtttcctgaatacagcacactgatgggtcttgactctttatccaacttgccagtctgtgtcttttaattgcagaatttagtccatttatatttaaagttaatattgttatgtgtgaatttgatcctgtcattatgatgttagctggtgattttgctcattagttgatgcagtttcttcctagtctcgatggtctttacattttggcatgattttgcagcggctggtaccggttgttcctttccatgtttagcgcttccttcaggagctcttttagggcaggcctggtggtgacaaaatctgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgtggagaaataggaacacttttacactgttggtgggactgtaaactagttcaaccattgtggaagtcagtgtggtgattcctcagggatctagaactagaaataccatttgacccagccatcccattactgggtatatacccaaatgactataaatcatgctgtctataaagacacatgcacacgtatgtttactgcggcactattcacaatagcaaagacttggaaccaacccaaatgtccaacaatgatagactggattaagaaaatgtggcacatatacaccatggaatactatgcagccataaaaaatgatgagttcatgtcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacatgttagtgggtgcagcgcaccagcatggcacatgtatacatatgtaactaacctgcacaatgtgcacatgtaccctaaaacttagagtataataaaaaaaaaaaaattaaaaaaaaaaaaaaaacttttgctcaatagaaattaacttgttagctccggcctgtatgaaatacattaaaaatgaaattcttacagcctgcaaagtcttccaagggggtaaagttccacattatatcacacacatatgcatttttgtttgctttactgtgacttggatttactacagagacctctaatttcttaaatgacttttcacttatctacagatggaagaataggacacgtaaatgcatcactcataattgagtgtttgtaatacctctcacatcttgaatcaaaataagaaataaaatgctgaaactacaaaaaaaaaaaatacaacaaatagggccaattgatgtgagctgtagcaatcatggcaggcttcaaaaaggaggggtggggttcaggccgggcatggtggatcatgcctgtaatctcagtgctttcgaggccaaggtggagcaatcatttgaggccaggggttcaaggatgcggtgacctatgactgtgccgctgtactccattctgagtgacagaccaagaccctgtctcttaaaaaataaaatatgataataaatacacataaaaaaggaggtgcagtatggtctgggccttgaagggtgagtaggagttagaggaatattagagagggagaaaacattccaagcaggaacaggaatggcccatgtggagatgagtcagaagccgattggctgtagcagaagatgagtggtgggatttcctaagtcaggtggggccagctgtgagatggaaggaggaatgttaaggatccaagagagtgttgttgagatctgatgcagcaagcattagatttctgataggtccttgaatgaagatgtgataggatgcgacttggtcatgacagcatcagctctgtactctagaaagggtccctcaggcagggacatttgctccccagaggtggagaagcagagtgcatgagaagggttcttactacattgcatttgtacctcaacagcctccaatctctctttcagattgtgtaccgcggggatccctatagtgcacacaactccacagagtttctcatctacgaaagaggagtcgaggttggatgttggggcttgtgcatcaactccgtgttttcctcactttattcttgtaagtctttctctctcctaaggatgtcttctaaaagtttctggtctagcacaacttggatttgatatttctatgtgtatttcctactgtttaaaataccagattcttaataaatctatggacgtcaggaaaaactatcaccttaaaaaggaccatataaagcatccttttttcatcatgaattctaggtgtcatttcctcactgattactttgctcttggaatcagtgggttcctgatcaagtttctctggaaaatgaaagggaatctttacaagatgaagtgttggatggatgagtcccagcaggttcgtgttctactggagcaagatcattggtagcatttcatagataggcttatcagatttgtagatgatacagaagagaagcaatattaatatgacaggtgacaaagtcagcatgcaaaatgaccttagctcagatgtgtaactgggtgggttataggagcaccacaccaggattggggacagctggctttccttcccaggtgccagcttttctgagcctcttaacctccacttacctaagttgcagctgtgaagacactgaacaaagggatttctgctattgtctttatgtaagtccaggtggctgtgctataactccagggcattgcacaggctgcattgtacttgcgactgtgtatggatatctccaagctctgtatacattgagttaagtgaaacgtctggcctgaaaaatgtatgcagttctcctaattataaagagctcctataaagagctcttcttctgcaagattgtaagaataactgacccccaattcagggctctgggctttggacaggtccaggggacagcagggatatggcccaggtgaaggaagtgggaagagatggggagaatccctgttaccattctcaggaggactcactctgtcactgtcagcctgcttcttgtgaagggaaagtgtggatgtatgtgagggccccttccccttccctttacctagccatagcacagcaggaaacctgcctcaagacctcttaaaaatttagcccaattgggaaaatgggacctcatataaacatgcaagttgatgagcccaataatcacgtaacaacaacattcattattttaaaatttatgatactcaaagagtagtgcttaaagatcacatttatttagcaaactcttttcttcctcttcgagtgtgcaaactaagaaaaaatccaggaggaagatttcaagcacttaaaatagaataaaagaatgttagactaaaagggtctagtaaattatccagcttaagtccttcatgatttcaagggctgaggggactatttaccactaccctcatgcttttttttgtttttcttctttttttaagacaaagtctcactctgccacctgagctggagtgcagtggcatgatcatggctcactgcagcctcaaccttctgggctcaagcaagtcccagccctctgagtacccgggaccacaagcacatgccactgtgcctggcttattaatttaaaaaaaattttttttttagagatggggtctccctacattgcccaggctggtctcgatctcctgggctcaagtgatcctcctgtctcagcctcccacagtgctgggattacaggcatgaggcactacatccagccctaccttcatctttgtcagaaagaaaagtgtcaatctaaacttatttttcctcagggaactcctactagataacattccattagtcttgtccgcataaattttatggcatttgaaactttaaagcatattatttttaggtcccttctagttaaaagcgtttctagttacaaaagaaattattagttgaatagttttccaaagttttattaatgtataaatctatgggatccctaccattgttaattcaaatcaacattttatttataaaataccagccagctctgctcaaggattttagggattccccatgcccataggataaagtcaaattcctgagcttttcacagtttggcctagttcatactttgccctttttcactcccatatcaattaaccaccctcaatgacccactatgcagactttctcatttctgggtatttgccactgcttttagtttcttctgcctgaactgctctttctacttccccaactggacaacttctaataatatttttagacccagcttaaatattacttcctcagtgaaatcattcaagttttcttccccctgcacctcaacccactctgggcagaatcaatcctttcctttccttccagagctcactatattaccctctattttggattcatcacataacatcaggagcagattcagagtttctatccacatggaggttggcaggacaatctggctgctgaggtggagagtgctgatagggacttgtttctaagctccttttgtatacagcaggtacaggtataccaattagaacaacaacactgagtactaagatatggcctattgttcataccataatatcatacactactagtcataactggtgcttaaatctataatttcccctcagtcctgaagagaggcattgtgtgaggatatgactaccactgggtatcagacccatcagagggcaattcaagcttggccctgaagttcactgaattatctttgtgaataaagtattctttgccagactttcagtctctgtactgaagttacttatgtttccaaatacctctttgccctactttgccttctcctctgctggcatacacacttggataagagcatttccctaatgggtattgatggtatcttttaggtattgagacaccttagattacattttctgtataaatataattattaagcaagcagaaactgttctagctttattctcatttttgtattctcaaccttaacaacagtcctccagtcatggtataagtttaatagaggaagcaatgttgcctacttgtcatttccagatctttgtcttccatcagtgtgagagtgtttagtttcataattgattcaaatgtactgcaaataaggttgcttgtctacaaggtgtaagtccaataattatatgaatagatatggaaaatgtcattcttaatagtcacgtttcaagtatctgttatttatcctctgattcttgtgttccaggagaatggggcagggaagagatgaggaatgtgtggattaaaaacgactgagggaagaagataaaaacaatttagagattttccttgccattgtagaaaaccttgtaaaatgttttaaagcagaatttttcatttttaaaaattagttctgccactatactgatataagcaagccaagttgacctgctagaagaggaacccagttaaaagtcaactgtctccagctcagcctgtcttgttgcttaattggatgttttctacatgctaggcattaagctgggtgatttcatagacacttcatctcacttgatcctggcaacaactccaaaagatggttttattattcccagttttcagatgaggaagctggggttttgagaagtcaaggaacttgtttaccaagagttagggagccagcgatgaggagatgctgggtgctgtgggagccccttaccagtctgggaaggattggcaagtcagggaaggtgtcttcagataattgtacatcttttacctccttctcaggtgtgcagactccattcagatgggggctaagaaatcatgaatgacttagatcattctgtaaattttggttgtttcaattttccccaacctgactttgggttgtagggccaagataggcctgtgttattattttcgtcctatggggccacagcccatctgatttagaataacccacacttttcttgaagccccaggttcccatagttaggtcagattttccaccacctttctcgcttctttttgctatccagctgtctgctaaacagtgccagattcttctttgtagaaaaaaaatcagcctaccaaggcaatttcaagctgtcctttatggaaaggtctttaaaatggttccaaaagccagctgttttcaacccacagataaggggattcttttgttcctggctcagtatcaaaggatgtctaaaaaggtgaatcttcagaagaaaggattgtctgaaattattaaatgttatgaggcactgccagctgtaatttctccctctcttcttcccacagactttcagaaagttttggtatcctacattggattaaagggtctttacttcacgggatatttgctgtttggcctggggacgggatttattgggctcttcccgaatgtctactccaccctggtcctgtgcagcctgtttggtgtaatgtccagcaccctgtacactgtgccctttaacctcattactgagtaccaccgcgaggaagaaaaggaggtgtgctgtcattgaacctctgccttgggtaacatccagactcatctgaaggctggggaggatttggtagcaatgtgtagagcagagctggaaaataagggttcttgaaacagcaacagtcaaatgcccaaccaactgcaaccatgatctggtacaggcatgctcaccagcccagcccagcccagctgtcctgtcctccttattctcaactcctgctcacttagggagctgtagggcaagaagtgttactcccatttttctaattagaaaaaggcttggtgaggctgagaaaccagcccacaattacacaaagacaaaaaaagggggacccaggtcttctgtaaccacctcataggtctccctggtgccacctgcagcatttctagataccgacaataagggtctccctatccccaactttcagaggtacatagtggaatttcttggcattatttgctgagggtaggaaagtcctaggcaatggagtccagattttagtaagtcagaccccatgtgtgagaaggaaagctggaatcatgggtggtaatccccactttgctgccctgagcgggacctgtgccatttaaaccttggaacgcaactagtgaacaagcgcctatgcagtgcttataacagagttggtgtttccactctaaaattaccacctcttcttatcaaaatttgcaactttcttcctgtgaaaataggcttatagttccttaactggagcaatgatataaaagctacagagataactccaagtttaatccagctcagcatgaaactgcaaaaacactgaacatataaccatcagactataattctggccatgtttttcatcatttgatctggcagcagggcagaggtgcaccaggagtaaaaacctttcaaaaaagaggtgtggctttcctcatcccctggcaaacattgtcaccaaggtgggatgttgctgctaatgggagagtcatcataaaaaactgtccctacaaggagagtgttacatggtgttgggggaagaaaacctacgcttcaggctcagagaaacctggattcaaatcctgcctctctcacttggaagctgtgttacactggggatgttatttacttgtttattagttagcacttagaatgagttcatttttaggtgagataatatattcacaatgatacacaaaagtgcacactgtgtgctccctttaccatttactatctccctccagccccaacgtgttaccactcttactagttttttgttaatctttccggcatttttaaataaaaaccaagcaaatgtaaatctgttttcttattttcctttcttattcaaaggataatatgcgtttttcagcataatgctttttttttgcctaatagtgtaggaaatcatcatttaagctcgcaggccctcagtttcctcatgaggcaaatggaaagaatgttatttaccttatagaaaagcaaacaaattagtggatgtgcttagcatagtgccccctagcacccgacaaagactcaaaatgtgatagctagtattataattgttaccatgggggtaatgataataacatgtgcatccagggaggtggtgaggttgaaatacagtaacaggtaaaaaaaaaacccaggcccagcacataagcgatacccaacaaatgacaaagttatcagccagttcggaagaaatatcaagtattaaccaaacattattatattaatcaagtagcaaccatattttgaaagattgaaatgctttttaaagttgcttctcttaaggacatcagcaattcaacatccttcttaaaatctttgaatttttaacactaagaaaggaaaagaatgggatatttattaagtatccactacattgtattgattatggcccgccacttgtttttgtacagtctgcgagctaagaattccttttaattgtttaaatgattgaaaaaaactttcaaaagaaggttaatatcttgtgacaggtgaaaattacatgaaatcaaatgctaatacccatcaatattcagttcagttccacatcaccaattcatgaaaattacatgaaatcaaatgctaataatgtccatcaatatatattggaaagccaactcagctccacattgcccctgcttttgcactacagcagcagagttgagtagttatgaccagggctgtataatccacgaagccaaaggtatttactatctggctggtcacagaaaaattttgctgacctgtgccctaaatgacagttccttgtaggtcaaatggtggttctgtgcttcctttgcagaggcagcaggccccaggaggggacccagacaacagcgtgagagggaagggcatggactgcgccaccctcacatgcatggtgcagctggctcagatcctggtcggaggtggcctgggctttctggtcaacacagccgggaccgttgtcgtcgtggtgatcacagcgtctgcggtggcactgataggctgttgctttgtcgctctctttgttagatatgtggattaggtcaataaagagacaatgaccctaacctcaga

SEQ ID NO: 2 - DNA, 601 bp, Homo sapiens
  misc_feature (301)..(301): N= C or G
ttccctttcattttccagagaaacttgatcaggaacccactgattccaagagcaaagtaatcagtgaggaaatgacacctagaattcatgatgaaaaaaggatgctttatatggtcctttttaaggtgatagtttttcctgacgtccatagatttattaagaatctggtattttaaacagtaggaaatacacatagaaatatcaaatccaagttgtgctagaccagaaacttttagaagacatccttaggagagagaaagacttacaagaataaagtgaggaaaacacggagttgatgcanaagccccaacatccaacctcgactcctctttcgtagatgagaaactctgtggagttgtgtgcactatagggatccccgcggtacacaatctgaaagagagattggaggctgttgaggtacaaatgcaatgtagtaagaacccttctcatgcactctgcttctccacctctggggagcaaatgtccctgcctgagggaccctttctagagtacagagctgatgctgtcatgaccaagtcgcatcctatcacatcttcattcaaggacctatcagaaatctaatgcttgctgcatcagatct

SEQ ID NO: 3 - protein, 460 aa, Homo sapiens
MGSNSGQAGRHIYKSLADDGPFDSVEPPKRPTSRLIMHSMAMFGREFCYAVEAAYVTPVLLSVGLPSSLYSIVWFLSPILGFLLQPVVGSASDHCRSRWGRRRPYILTLGVMMLVGMALYLNGATVVAALIANPRRKLVWAISVTMIGVVLFDFAADFIDGPIKAYLFDVCSHQDKEKGLHYHALFTGFGGALGYLLGAIDWAHLELGRLLGTEFQVMFFFSALVLTLCFTVHLCSISEAPLTEVAKGIPPQQTPQDPPLSSDGMYEYGSIEKVKNGYVNPELAMQGAKNKNHAEQTRRAMTLKSLLRALVNMPPHYRYLCISHLIGWTAFLSNMLFFTDFMGQIVYRGDPYSAHNSTEFLIYERGVEVGCWGFCINSVFSSLYSYFQKVLVSYIGLKGLYFTGYLLFGLGTGFIGLFPNVYSTLVLCSLFGVMSSTLYTVPFNLITEYHREEEKEVCCH

SEQ ID NO: 4 - DNA, 601 bp, Homo sapiens
  misc_feature (301)..(301): N= C or T
tgctctgagaggagtaagagacaggaggaccagaggaaaatgcagacatttttctttgagatataaaattttaattccaagggatgatagctacgggggatttgggaattcagttagaccccatgaaactcttctcgtcaaacagacaaaacaaacaattaaaaaaacaacaacaacaaagagcaagaatattttcccttgtaaagaaaaaatgttgcatctttacctgttcagcatgatttttgttttttgctccctgcattgccagctctggatttacgtaaccatttttaactttctngatagaaccatactcgtacattccatctgatgacaatggagggtcctgaggggtttgctgtgggggaatgccctttgcaacctctgtaagtggggcttcagagatactgcacagatgaacagtaaaacacaaagtgagcaccaatgcagagaagaagaacatgacctggaattctgtacccaacagtcttcccagctccagatgggcccagtctatagcacccaaaaggtaacccagggcacctccaaaacctggaaagcaagaaaagctatgttagcatatttagcaaattatcgttca

SEQ ID NO: 5 - protein, 317 aa, Homo sapiens
MAVQGSQRRLLGSLNSTPTAIPQLGLAANQTGARCLEVSISDGLFLSLGLVSLVENALVVATIAKNRNLHSPMYCFICCLALSDLLVSGSNVLETAVILLLEAGALVARAAVLQQLDNVIDVITCSSMLSSLCFLGAIAVDRYISIFYALRYHSIVTLPRARRAVAAIWVASVVFSTLFIAYYDHVAVLLCLVVFFLAMLVLMAVLYVHMLARACQHAQGIARLHKRQRPVHQGFGLKGAVTLTILLGIFFLCWGPFFLHLTLIVLCPEHPTCGCIFKNFNLFLALIICNAIIDPLIYAFHSQELRRTLKEVLTCSW

SEQ ID NO: 6 - DNA, 51 bp, Homo sapiens
  misc_feature (26)..(26): N = A or C
tcatctgctgcctggccttgtcgganctgctggtgagcgggagcaacgtgc

SEQ ID NO: 7 - DNA, 601 bp, Homo sapiens
  misc_feature (301)..(301): N = T or G
gtgagcttggtggagaacgcgctggtggtggccaccatcgccaagaaccggaacctgcactcacccatgtactgcttcatctgctgcctggccttgtcggacctgctggtgagcgggagcaacgtgctggagacggccgtcatcctcctgctggaggccggtgcactggtggcccgggctgcggtgctgcagcagctggacaatgtcattgacgtgatcacctgcagctccatgctgtccagcctctgcttcctgggcgccatcgccgtggaccgctacatctccatcttctacgcactgngctaccacagcatcgtgaccctgccgcgggcgcggcgagccgttgcggccatctgggtggccagtgtcgtcttcagcacgctcttcatcgcctactacgaccacgtggccgtcctgctgtgcctcgtggtcttcttcctggctatgctggtgctcatggccgtgctgtacgtccacatgctggcccgggcctgccagcacgcccagggcatcgcccggctccacaagaggcagcgcccggtccaccagggctttggccttaaaggcgctgtcaccctcaccatcctgctgggcattttct

SEQ ID NO: 8 - DNA, 601 bp, Homo sapiens
  misc_feature (301)..(301): N = C or T
gtggccaccatcgccaagaaccggaacctgcactcacccatgtactgcttcatctgctgcctggccttgtcggacctgctggtgagcgggagcaacgtgctggagacggccgtcatcctcctgctggaggccggtgcactggtggcccgggctgcggtgctgcagcagctggacaatgtcattgacgtgatcacctgcagctccatgctgtccagcctctgcttcctgggcgccatcgccgtggaccgctacatctccatcttctacgcactgcgctaccacagcatcgtgaccctgccgngggcgcggcgagccgttgcggccatctgggtggccagtgtcgtcttcagcacgctcttcatcgcctactacgaccacgtggccgtcctgctgtgcctcgtggtcttcttcctggctatgctggtgctcatggccgtgctgtacgtccacatgctggcccgggcctgccagcacgcccagggcatcgcccggctccacaagaggcagcgcccggtccaccagggctttggccttaaaggcgctgtcaccctcaccatcctgctgggcattttcttcctctgctggggccccttcttcctgc

SEQ ID NO: 9 - DNA, 51 bp, Homo sapiens
  misc_feature (26)..(26): N = G or C
cctcatcatctgcaatgccatcatcnaccccctcatctacgccttccacag

SEQ ID NO: 10 - DNA, 3115 bp, Homo sapiens
agacgcagtcttcagcaaggaagtgctgggaacgccctggagtgaacccaggaagatgcctgcagtgggtgccagggcccctctccaccgtccctgctgggcttcggggccacgcccgactgctgtgaacggcctgcggagcaccacgtgcgacggctggaggcgagaggtctgcctttgatgtggctgttggtgcagggcctgtggtgccttccgcagcggaaatggcgcgccgcccggggagggcgggagcagcgtcccgggtgcccctgtgaggatgagcgacgagatgactggagggtccctgaagacctcactagggtgcccccagccggtccgctcccaggaagcgacacccccacagccccagggctgcagctgagggggtcgccactctggctgggcgaggctgggcccttgggggcaggcgccagagtggcctcaggctctacaagatgcctgaaaacaccaacctctccagggctcactagcattggacgctttcacgctctgccctggccggaagccccctcaccccgcgcgatgtgcaaactcctgcagggctcactcagtttccagaactttaattattggaaagttctccctggtccagcccccaaatctgccgtgaacgttgacagctgagttgctgctccatgcgtgctttggctgagagcagaggggacccctgtcctccctgagctgctgacgaggggaggggtgaagggtggggcctctggagagggcaggtcccggggaagctccggactcctagaggggcggccaggtgggggccctggtgaccaggacagactgtggtgttttttaacgtaaaggagatccgcggtgtgagggaccccctgggtcctgcacgccgcctggtggcaggccgggccatggtgggtgctcacgcccccggcatgtggccgccctcagtgggaggggctctgagaacgactttttaaaacgcagagaaaagctccattcttcccaggacctcagcgcagccctggcccaggaaggcaggagacagaggccaggacggtccagaggtgtcgaaatgtcctggggacctgagcagcagccaccagggaagaggcagggagggagctgaggaccaggcttggttgtgagaatccctgagcccaggcggtagatgccaggaggtgtctggactggctgggccatgcctgggctgacctgtccagccagggagagggtgtgagggcagatctgggggtgcccagatggaaggaggcaggcatgggggacacccaaggccccctggcagcaccatgaactaagcaggacacctggaggggaagaactgtggggacctggaggcctccaacgactccttcctgcttcctggacaggactatggctgtgcagggatcccagagaagacttctgggctccctcaactccacccccacagccatcccccagctggggctggctgccaaccagacaggagcccggtgcctggaggtgtccatctctgacgggctcttcctcagcctggggctggtgagcttggtggagaacgcgctggtggtggccaccatcgccaagaaccggaacctgcactcacccatgtactgcttcatctgctgcctggccttgtcggacctgctggtgagcgggagcaacgtgctggagacggccgtcatcctcctgctggaggccggtgcactggtggcccgggctgcggtgctgcagcagctggacaatgtcattgacgtgatcacctgcagctccatgctgtccagcctctgcttcctgggcgccatcgccgtggaccgctacatctccatcttctacgcactgcgctaccacagcatcgtgaccctgccgcgggcgcggcgagccgttgcggccatctgggtggccagtgtcgtcttcagcacgctcttcatcgcctactacgaccacgtggccgtcctgctgtgcctcgtggtcttcttcctggctatgctggtgctcatggccgtgctgtacgtccacatgctggcccgggcctgccagcacgcccagggcatcgcccggctccacaagaggcagcgcccggtccaccagggctttggccttaaaggcgctgtcaccctcaccatcctgctgggcattttcttcctctgctggggccccttcttcctgcatctcacactcatcgtcctctgccccgagcaccccacgtgcggctgcatcttcaagaacttcaacctctttctcgccctcatcatctgcaatgccatcatcgaccccctcatctacgccttccacagccaggagctccgcaggacgctcaaggaggtgctgacatgctcctggtgagcgcggtgcacgcggctttaagtgtgctgggcagagggaggtggtgatattgtgtggtctggttcctgtgtgaccctgggcagttccttacctccctggtccccgtttgtcaaagaggatggactaaatgatctctgaaagtgttgaagcgcggacccttctgggtccagggaggggtccctgcaaaactccaggcaggacttctcaccagcagtcgtggggaacggaggaggacatggggaggttgtggggcctcaggctccgggcaccaggggccaacctcaggctcctaaagagacattttccgcccactcctgggacactccgtctgctccaatgactgagcagcatccaccccaccccatctttgctgccagctctcaggaccgtgccctcgtcagctgggatgtgaagtctctgggtggaagtgtgtgccaagagctactcccacagcagccccaggagaaggggctttgtgaccagaaagcttcatccacagccttgcagcggctcctgcaaaaggaggtgaaatccctgcctcaggccaagggaccaggtttgcaggagcccccctagtggtatggggctgagccctcctgagggccggttctaaggctcagactgggcactggggcctcagcctgctttcctgcagcagtcgcccaagcagacagccctggcaaatgcctgactcagtgaccagtgcctgtgagcatggggccaggaaagtctggtaataaatgtgactcagcatcacccaccttaaaaaaaaaaaaaaaaa

(19) United States
(12) Patent Application Publication
(10) Pub. No.: US 2011/0086805 A1
(43) Pub. Date: Apr. 14, 2011

(54) IN VITRO METHOD FOR DIAGNOSING SKIN CANCER

(75) Inventor: Nadem Soufir, Clamart (FR)

(73) Assignee: Assistance Publique - Hopitaux De Paris, Paris (FR)

(21) Appl. No.: 12/744,172

(22) PCT Filed: Nov. 21, 2008
(86) PCT No.: PCT/EP08/66028

§ 371 Date: May 21, 2010

(30) Foreign Application Priority Data
Nov. 21, 2007 (FR) 07/59193

(51) Int. Cl. A61K 38/00; C12Q 1/68; A61K 31/7088; A61P 35/00

(52) U.S. Cl. 514/18.6; 435/6; 514/44 R

(57) ABSTRACT

The present invention relates to an in vitro method for identifying an individual suffering from or having a predisposition to skin cancer, characterized in that it comprises the step of analysing a biological sample originating from said individual by a) detecting a polymorphism of the MATP/SLC45A2 gene (SEQ ID NO: 1), and/or analysing the expression of the MATP/SLC45A2 gene; to the use, for preparing a composition for the treatment and/or prevention of a skin cancer in an individual, of a compound which specifically increases the expression of the MATP/SLC45A2 gene in a skin cell; and to a method for the selection, in vitro, of a compound capable of being of use in the treatment of skin cancer.

The present application claims the priority of application FR0759193, filed on Nov. 21, 2007, which is incorporated to the present patent application by reference.

The present invention relates to the diagnosis of skin cancer and more specifically to an in vitro method for diagnosing a skin cancer for a subject suspected of having or being predisposed to a skin cancer.

Knowledge on genetic predisposition to melanoma has made considerable progress during the last fifteen years. Two important genes of predisposition to melanoma have thus been identified, which genes correspond to CDKN2A and CDK4. Both of these genes are essentially involved in family predisposition and in multiple sporadic cases of melanomas (HUSSUSSIAN et al., Nat. Genet., Vol. 8, p: 15-21, 1994); KAMB et al., Nat. Genet., Vol. 8, p: 23-6, 1994; ZUO et al., Nat. Genet., Vol. 12, p: 97-9, 1996).

Recent studies have identified major loci involved in the susceptibility on the 1p22 (GILLANDERS et al., Am. J. Hum. Genet., Vol. 73, p: 301-13, 2003) and 9q21 (JONSSON et al., J. Natl. Cancer Inst., Vol. 97, p: 1377-82, 2005) chromosomes.

However there exist today recurrent pieces of evidence on an implication of multiple genetic factors in the predisposition to melanoma and the identification of other genetic variants associated with a lesser contribution to the melanoma risk is in progress. Among these new variants, mention may thus be made of the variants of the RHC type of the MC1R gene, for which it has been shown that it forms a gene of melanoma with low penetrance (PALMER et al., Am. J. Hum. Genet., Vol. 66, p: 176-86, 2000; KENNEDY et al., J. Invest. Dermatol., Vol. 117, p: 294-300, 2001; MATICHARD et al., J. Med. Genet., Vol. 41, p: e13, 2004; STRATIGOS et al., J. Invest. Dermatol., 2006; LANDI et al., J. Natl. Cancer Inst., Vol. 97, p: 998-1007, 2005; DEBNIAK et al., Int. J. Cancer, Vol. 119, p: 2597-602, 2006); HEALY, Photodermatol. Photoimmunol. Photomed., Vol. 20, p: 238-8), 2004) and that it acts as an element modifying the melanoma risk in individuals bearing mutations of CDKN2A (BOX et al., Am. J. Hum. Genet., Vol. 69, p: 4, 2001; VAN DER VELDEN et al., Am. J. Hum. Genet., Vol. 69, p: 4, 2001; CHAUDRU et al., Cancer Epidemiol. Biomarkers Prev., Vol. 14, p: 2384-90, 2005). Five allelic variants of MC1R (D84E, R142H, R151c, R160W and D294H) have thus been identified as being strongly associated with a phenotype characterized by red hair, pale skin, freckles and sensitivity to the sun and are now known under the term of RHC variants.

However, there still exists a significant need for identifying other genes involved in the susceptibility to skin cancer in order to clearly and completely identify subjects having the greatest predisposition to melanoma so as to monitor these subjects as a priority.

The present invention is based on the discovery of a strong association between certain alleles of the MATP/SLC45A2 gene and melanoma and more specifically between the allele SLC45A2 374F and increased melanoma risk, while the allele SLC45A2 374L has a protective effect towards melanoma. Further, it was first demonstrated that the SLC45A2 374F allele was associated with a reduction in the amount of photoprotective melanin (eumelanin) in the epidermis (NORTON et al., Mol. Biol. Evol., 24: 710-22; 2007), which suggests either a reduction in the expression, or a function loss of the MATP/SLC45A2 protein. From the results obtained by the inventors, it is therefore also possible to conclude that the presence of the allele 374F of the MATP/SLC45A2 gene is also associated with an increased melanoma risk, and that the presence of the SLC45A2 374L allele is associated with a protective effect against the melanoma.

By “MATP/SLC45A2 gene” is meant here the gene coding for the transporter protein associated with membranes (MATP) originally called AIM1, and recently renamed as “member 2 of the family of solute transporters” (or SLC45A2). The gene MATP/SLC45A2 is located on the 5p chromosome (gene ID 51151) and has the sequence as described in the accession number NT_{—}006576.15 (SEQ ID NO:1). The MATP/SLC45A2 protein (mRNA sequence: accession number NM_{—}001012509) has the protein sequence SEQ ID NO:3 (accession number: NP_{—}001012527).

The protein MATP/SLC45A2 is designated herein equally by the terms of “MATP”, “SLC45A2” or “MATP/SLC45A2”.

Because of its overexpression established in certain melanoma cell lines relatively to its expression in normal melanocytes (absence of expression in normal tissues), the gene MATP/SLC45A2 was known as a melanoma antigen (Harada M et al., Cancer Research, 1089-1094; 2001).

The present invention is based on the fact that the allele SLC45A2 374F is associated with increased melanoma risk, while the SLC45A2 374L allele on the contrary has a protective effect towards melanoma.

The polymorphism SLC45A2 F374L was already known at the date of the invention. This polymorphism had actually been described in the prior art as one of the many polymorphisms identified in individuals suffering from oculocutaneous albinism. In such patients, this variant seems to be rather common and 8.5% of the tested individuals have this variant heterozygotously (Rundshagen U. et al., Human mutation; 23:106-110; 2004). The parallel between this polymorphism and the loss of pigmentation related to albinism is however not suggested. Also, Newton et al. (Am. J. Hum. Genetic; 69: 981-988; 2001) have identified this polymorphism in individuals, including in individuals with normal pigmentation and this homozygotously. According to the latter article, the mutation F374L of the SLC45A2 protein is “neutral” towards hypopigmentation mechanisms responsible for albinism.

Moreover, other studies have shown the association between the F374L allele of MATP/SLC45A2 and normal human pigmentation in certain populations, notably in Afro-Americans (Norton H L et al., Mol. Biol. Evol. 24 (3): 710-722, 2007; Graf et al., Hum. Mutat. Vol. 25, p: 278-84, 2005). However, this association could not suggest the present invention, since several hundred genes influence pigmentation without however being associated with melanoma predisposition (see for example Sulem P, Nature Genetics; Vol. 39, No. 12, 2007).

Thus, the present inventors were moreover able to show that although it is associated with pigmentation, the MATP/SLC45A2 374 variant identified here predicts the melanoma risk independently of pigmentary characteristics. More particularly, the present inventors have shown that the SLC45A2 L374F effect on melanoma predisposition persists even after stratification on the pigmentation characteristics or in a logistic regression model integrating the 2 genetic risk factors (MATP F374L, MC1R variants) and clinical risk factors (color of the eyes, of hair, phototype and number of naevi). This suggests that in view of the melanoma risk, the information related to the genotype SLC45A2 L374F is not redundant with the pigmentation characteristics, and that this variant is therefore a strong and independent melanoma risk factor (see also the article of the inventors Guedj M et al., Human Mutation 29 (9), 1154-1160, 2008).

Accordingly, a first object of the invention relates to an in vitro method intended to identify an affected subject or having predisposition to skin cancer, characterized in that it comprises the step for analyzing a biological sample from said subject by:
(a) detection of a polymorphism of the MATP/SLC45A2 gene (SEQ ID NO:1), and/or
(b) analysis of the expression of the MATP/SLC45A2 gene.

As used herein, the term of “subject” refers to a mammal, preferably to a human.

As used herein, the term of “biological sample” refers to any solid or liquid sample from a subject. As an example of solid samples, mention may be made of a skin sample and, as an example of liquid sample, mention may be made of a blood sample.

Preferably, said biological sample is a blood sample when a step for detecting a polymorphism of the MATP/SLC45A2 gene is carried out.

Still preferably, said biological sample is a skin sample when a step for analyzing the expression of the MATP/SLC45A2 gene is carried out.

As used here, the term of “skin cancer” refers to a cancer involving a cell type of the epidermis or of the dermis, preferably said skin cancer is a melanoma.

Advantageously, said polymorphism of the MATP/SLC45A2 gene associated with predisposition to skin cancer corresponds to a polymorphism associated with a single nucleotide (SNP).

As an example of such SNPs, mention may be made of the SNP rs16891982 (an N nucleotide in position 301 of SEQ ID NO:2, the G nucleotide being associated with skin cancer) and resulting in the presence of phenylalanine in position 374 of the protein MATP/SLC45A2 (SEQ ID NO:3) and SNP rs26722, (N nucleotide in position 301 of SEQ ID NO:4, the C nucleotide being associated with skin cancer) resulting in the presence of a glutamate residue in position 272 of the MATP/SLC45A2 protein (SEQ ID NO:3).

Still advantageously, said polymorphism of the gene MATP/SLC45A2 is associated with a significantly lower expression level of the MATP/SLC45A2 gene.

Preferably, said SNP corresponds to the SNP rs16891982 (N nucleotide in position 301 of SEQ ID NO:2, the G nucleotide being associated with skin cancer) and resulting in the presence of phenylalanine in the position 374 of the MATP/SLC45A2 protein (SEQ ID NO:3).

One skilled in the art considering his/her general knowledge and the present description will be able to simply identify other polymorphisms of the MATP/SLC45A2 gene associated with predisposition to skin cancer.

In a preferred embodiment, the method of the invention allows identification of an affected subject or having predisposition to skin cancer by analyzing, in addition to one of the polymorphisms of the MATP/SLC45A2 gene and/or its expression, a polymorphism of another gene of susceptibility to melanoma.

This other susceptibility gene is for example the MC1R gene (gene coding for the receptor to melanocortin 1). The present inventors could actually show that four polymorphisms of the gene of the receptor to melanocortin 1, or MC1R(NP_{—}002377.4, SEQ ID NO:5), are associated with increased melanoma risk.

The MC1R protein is coded by the human gene MC1R (gene ID 4157), which is transcribed into an mRNA of sequence NM_{—}002386.3 (SEQ ID NO:10). Five allelic variants of MC1R (D84E, R142H, R151c, R160W and D294H) were identified as being strongly associated with a phenotype characterized by red hair, pale skin, freckles and sensitivity to the sun. These allelic variants are now known under the term of “RHC variants” (FLANAGAN et al., Hum. Mol. Genet., Vol. 9, p: 2531-7, 2000; DUFFY et al., Hum. Mol. Genet., Vol. 13, p: 447-61, 2004; REES, Am. J. Hum. Genet. Vol. 75, p: 739-51, 2004; Sulem P, Nature Genetics; Vol. 39, No. 12, 2007).

These variants have already been associated with the melanoma risk in multiple populations (STURM et al., Pigment Cell Res., Vol. 16, p: 266-72, 2003; REES, Annu. Rev. Genet. Vol. 37, p: 67-90, 2003), and have all been identified as generating a loss of function in functional studies (BEAUMONT et al., Hum. Mol., Genet., Vol. 14, p: 2145-54, 2005; BEAUMONT et al., Hum. Mol. Genet., 2007). The inventors have again shown here that certain of these SNPs of MC1R are associated with increased melanoma risk, and this independently of the polymorphisms of MATP/SLC45A2 identified earlier.

In this embodiment, the method of the invention is an in vitro method intended to identify an affected subject or having predisposition to skin cancer, characterized in that it further comprises, c) the detection of a polymorphism of the MC1R gene (SEQ ID NO:10).

In a still more preferred embodiment, the detection of the polymorphism of the MC1R gene associated with predisposition to skin cancer, corresponds to several SNPs preferably selected from the group comprising the SNP rs1805006 (N nucleotide in position 26 of SEQ ID NO:6 (ss2425919), the nucleotide A being associated with skin cancer) and resulting in the presence of a glutamate in position 84 of the MC1R protein (SEQ ID NO:5), the SNP rs1805007 (N nucleotide in position 301 of SEQ ID NO:7, the T nucleotide being associated with skin cancer) and resulting in the presence of a cysteine in position 151 of the MC1R protein (SEQ ID NO:5), the SNP rs1805008 (N nucleotide in position 301 of SEQ ID NO:8, the T nucleotide being associated with skin cancer) and resulting in the presence of a tryptophan in position 160 of the MC1R protein (SEQ ID NO:5), the SNP rs1805009 (N nucleotide in position 26 of SEQ ID NO:9, the C nucleotide being associated with skin cancer) and resulting in the presence of a histidine in position 294 of the MC1R protein (SEQ ID NO:5).

Techniques for identifying a polymorphism of the gene MATP/SLC45A2 or MC1R are well-known to one skilled in the art and notably include the length polymorphism of the restriction fragments (RLFP), hybridization techniques, DNA sequencing techniques, resistance to exonucleases, microsequencing, solid phase extension using ddNTPs, solution extension by using ddNTPs, oligonucleotide ligating methods, methods for detecting SNPs such as specific allele dynamic hybridization, ligase chain reaction (LCR), mini-sequencing, the use of DNA chips or further specific allele oligonucleotide hybridization as a complement to a probe having simple or double labelling and by means of PCR reactions.

Preferably, said technique for identifying a polymorphism of the MATP/SLC45A2 or MC1R gene is a technique with which permits to detect a polymorphism associated with a single nucleotide (SNP).

The analysis of the expression of the MATP/SLC45A2 gene may be carried out by means of one of the many methods well-known to one skilled in the art and allowing the detection of the expression product of said gene such as its RNA or its protein product.

In a preferred embodiment, the expression of the MATP/SLC45A2 gene is carried out by analysis of the expression of mRNA transcripts, or mRNA precursors, such as a native RNA, of said gene. Said analysis may be carried out by preparing the mRNA/cDNA from cells of a biological sample from a patient, and by hybridization of the mRNA/cDNA with a reference polynucleotide. The prepared mRNA/cDNA may be used in an analysis by hybridization or amplification which includes, without being limited thereto, Southern and Northern analyses, PCR (polymerase chain reaction) analyses, such as quantitative PCR (TAQMAN) and the use of probes (probe arrays) such as DNA templates GENECHIP® (AFFYMETRIX).

Advantageously, the analysis of the expression of the mRNA level transcribed from a MATP/SLC45A2 gene involves a method for amplifying nucleic acids, such as for example RT-PCR (an experimental embodiment described in U.S. Pat. No. 4,683,202), ligase chain reaction (BARANY, Proc. Natl. Acad. Sci. USA, Vol. 88, p: 189-193, 1991), self sustained sequence replication (GUATELLI et al., Proc. Natl. Acad. Sci. USA, Vol. 87, p: 1874-1878, 1990), the transcriptional amplification system (KWOH et al., Proc. Natl. Acad. Sci. USA, Vol. 86, p: 1173-1177, 1989), Q-Beta Replicase (LIZARDI et al., Biol. Technology, Vol. 6, p: 1197, 1988), rolling circle replication (U.S. Pat. No. 5,854,033) or any other method for amplifying nucleic acids, followed by a step for detecting the amplified molecules by techniques well-known to one skilled in the art. These detection methods are particularly useful for detecting molecules of nucleic acids in very small amounts. As used here, the amplification primers are defined as being a pair of molecules of nucleic acids which may respectively pair to the regions 3′ and 5′ of a gene in a specific way (positive and negative strand, or vice versa) and surround a short region of said gene. Generally, the amplification primers have a length from 10 to 30 nucleotides and allow the amplification of a region with a length comprised between 50 and 200 nucleotides.

In another preferred embodiment, the measurement of the expression of the MATP/SLC45A2 gene is conducted by analyzing the expression of the protein translated from said gene. Said analysis may be performed by using an antibody (for example a radiolabeled antibody, labeled with a chromophore, a fluorophore, or an enzyme), an antibody derivative (for example an antibody conjugate with a substrate or with a protein or a ligand of a protein of a ligand/protein pair (for example biotin-streptavidin)) or an antibody fragment (for example an antibody with a single chain, a hypervariable domain of an isolated antibody, etc.) which specifically binds to the protein translated from the MATP/SLC45A2 gene.

Said analyses may be performed by many techniques within the reach of one skilled in the art including, without being limited thereto, immunological tests based on the use of enzymatic activity (enzyme immunoassay (EIA)), of immunological tests based on the use of radioactive isotopes (RIA), Western blot analysis and ELISA (enzyme linked immunoabsorbant assay) tests.

As an example of antibodies directed against the MATP/SLC45A2 protein, mention may be made of the antibodies available at ABNOVA CORPORATION or at SANTA CRUZ BIOTECHNOLOGY.

However, polyclonal antibodies may also be prepared by immunization of a suitable animal, such as a mouse, a rabbit or a goat, with the MATP/SLC45A2 protein (Homo Sapiens; SEQ ID NO:3) or a fragment thereof. The antibody concentration in the immunized animal may be monitored over time by standard techniques, such as an ELISA test, by using an immobilized polypeptide. At a certain time after immunization, for example, when the specific antibody titers are the highest, the cells producing the antibodies may be sampled from the animal and used for preparing monoclonal antibodies (mAc) by standard techniques, such as the hybridoma technique initially described by KOHLER and MILSTEIN (Nature, Vol. 256, p: 495-497, 1975), the technique of hybridomas of human B cells (KOZBOR et al., Immunol., Vol. 4, p: 72, 1983), the technique of EBV hybridomas (COLE et al., In Monoclonal Antibodies and Cancer Therapy, Alan R. Liss, Inc., p: 77-96, 1985) or the technique of triomas. The technique for producing hybridomas is well-known (see: Current Protocols in Immunology, COLIGAN et al., ed., John Wiley & Sons, New York, 1994). The hybridomas producing the desired monoclonal antibody are detected by screening the supernatants of hybridoma cultures for antibodies which bind to the polypeptide of interest, for example, by using a standard ELISA test.

The method according to the present invention may further comprise a step of comparing the expression level of the MATP/SLC45A2 gene in the biological sample from said subject with the expression level of said gene in a control sample.

As a control sample, it is notably possible to use a biological sample from a healthy subject, i.e. who is not affected or predisposed to skin cancer.

A significantly lower expression level than the expression level of the same gene in the control sample indicates that the patient is affected or predisposed to be affected by skin cancer.

A “significantly lower expression level of the MATP/SLC45A2 gene” refers to an expression level in a biological sample which is less than at least 20% of the normal expression level of said gene, preferably less than at least 50% of the normal expression level of said gene, and more preferably less than at least 90% of the normal expression level of said gene.

The “normal” expression level of the gene is the expression level of said gene in a control sample potentially corresponding to the biological sample from a patient not having skin cancer or preferably, to the average of the expression level of said gene in different control samples.

Another object of the present invention relates to the use, for preparing a composition intended for treating and/or preventing skin cancer in a subject, of a compound which specifically increases the expression of the MATP/SLC45A2 gene in a skin cell.

By skin cell is meant a cell of the dermis or of the epidermis. Preferably, said skin cell is a melanocyte.

Preferably, said compound specifically increasing the expression of the MATP/SLC45A2 gene is selected from the group comprising the MATP/SLC45A2 protein and its derivatives, a polynucleotide coding for such a protein or a vector comprising such a polynucleotide.

By MATP/SLC45A2 protein is preferably meant the MATP/SLC45A2 protein of Homo Sapiens (NP_{—}001012527; SEQ ID NO:3).

By “derivative” is meant a protein, the sequence of which has an identity percentage of at least 80%, for example at least 85%, preferably at least 90%, and more preferably at least 95% with the polypeptide sequence of the MATP/SLC45A2 protein.

By identity percentage between two polypeptide sequences, is meant the percentage of identical amino acids, between two sequences which have to be compared, obtained with the best possible alignment of said sequences. This percentage is purely statistical and the differences between two sequences are randomly distributed over the whole length of the amino acid sequences. By best possible alignment or optimum alignment, is meant the alignment with which the highest identity percentage may be obtained. Comparisons of sequences between two amino acid sequences are usually performed by comparing said sequences after their having been aligned according to the best possible alignment; the comparison is then performed on comparison segments so as to identify and compare similarity regions. The best alignment as possible for carrying out a comparison may be achieved by using the global homology algorithm developed by SMITH and WATERMAN (Ad. App. Math., Vol. 2, p: 482, 1981), by using the local homology algorithm developed by NEDDLEMAN and WUNSCH (J. Mol. Biol. Vol. 48, p: 443, 1970), by using the similarity method developed by PEARSON and LIPMAN (Proc. Natl. Acad. Sci. USA, Vol. 85, p: 2444, 1988), by using computer programs based on such algorithms (GAP, BESTFIT, BLAST P, BLAST N, FASTA, TFASTA, Genetics Computer Group, 575 Science Dr., Madison, Wis., USA), by using the multiple alignment algorithms MUSCLE (Edgar, Robert C., Nucleic Acids Research, Vol. 32, p: 1792, 2004). In order to obtain the best alignment as possible, the BLAST program will preferably be used with the matrix BLOSUM 62 or the matrix PAM 30. The identity percentage is determined by comparing both sequences aligned in an optimum way, said sequences may comprise additions or deletions with respect to the reference sequence so as to obtain the best alignment as possible between both of these sequences. The identity percentage is calculated by determining the identical position number between both sequences, by dividing the obtained number by the total number of compared positions and by multiplying the obtained result by 100 in order to obtain the identity percentage between both of these sequences.

By polynucleotide, is meant an RNA or DNA sequence, preferably said polynucleotide is a DNA sequence.

Still advantageously, said polynucleotide is operationally related to a gene expression sequence directing the expression of said polynucleotide in an eukaryotic cell, preferably in a skin cell. Said gene expression sequence corresponds to any regulatory sequence, such as a promoter sequence or a combination between a promoter sequence and an activator sequence facilitating transcription and effective translation of the polypeptide as described earlier. Said gene expression sequence may correspond to a constitutive or inducible viral or eukaryotic promoter sequence.

As an example of vectors comprising said polynucleotide, mention may be made of plasmids, cosmids, and viruses, notably adenoviruses and retroviruses.

Advantageously, said compound specifically increasing the expression of the MATP/SLC45A2 gene may be associated with a pharmaceutically acceptable carrier.

As an example of a pharmaceutically acceptable carrier, the composition may comprise emulsions, microemulsions, oil-in-water emulsions, anhydrous lipids, and water-in-oil emulsions, or other types of emulsions.

The composition according to the invention may further comprise one or more additives such as diluents, excipients, stabilizers and preservatives. Such additives are well-known to one skilled in the art and are notably described in “Ullmann's Encyclopedia of Industrial Chemistry, 6^{th} ed.” (various editors, 1989-1998, Marcel Dekker); and in “Pharmaceutical Dosage Forms and Drug Delivery Systems” (ANSEL et al., 1994, WILLIAMS & WILKINS).

Another object of the invention relates to an in vitro method for selecting a compound capable of being useful for treating skin cancer, characterized in that it comprises the steps of:
a) obtaining a cell expressing the MATP/SLC45A2 gene;
b) putting said cell into contact with at least one compound to be tested;
c) comparing the expression of the MATP/SLC45A2 gene between steps a) and b);
d) selecting the compound inducing an increase in the expression level of the MATP/SLC45A2 gene in the cell in step b) relatively to the expression level of said gene in step a).

Advantageously, said cell expressing the MATP/SLC45A2 gene is obtained from a subject having skin cancer.

Preferably, said cell has a significantly lower expression level of the MATP/SLC45A2 gene, as compared with a cell sampled on a subject not having skin cancer.

Preferably, said cell corresponds to a skin cell and more preferentially a cell stemming or derived from a melanoma.

As used here, the term of “compound” refers to any type of molecules such as polypeptides, polynucleotides, sugars, lipids or any other chemical compound.

The methods for determining the expression of the MATP/SLC45A2 gene are well-known to one skilled in the art. For example, the methods described earlier may be used.

The present invention will now be described in more detail with the examples which illustrate the invention without limiting the scope thereof by any means.

### EXAMPLES

### Materials and Methods

Study of the Population

1019 patients and 1466 control subjects, all of Caucasian origin, were prospectively included in the study between 2003 and 2006. The “Melanoma” patients were recruited between 2003 and 2006 in the melanoma cohort (MELAN-COHORT), a prospective cohort including all the patients with a melanoma for the Dermatological Department of all the Universities affiliated to the Paris Hospitals (Bichat, Percy, Ambroise Paré, Henri Mondor, Cochin and the Saint Louis Hospitals). The population study consisted in patients of 18 to 80 years of age with histologically proved malignant melanomas. The patients were not included if they were immunodeficient (HIV or transplantation), or if they suffered from genodermatosis predisposing them to skin cancer (albinism, Gorlin Syndrome, or pigmentary eptitheliomatosis). The control group consisted of 1466 Caucasian persons, without any skin cancer history.

The local medical ethics committee approved the study protocol. Enlightened consent was obtained from all the patients and control subjects registered for the study. After said informed consent, the genomic DNA was isolated from the peripheral blood leukocytes of all the participants by using the QIAAMP BLOOD MINI KIT (QIAGEN).

Collecting the Data on the Risk Factors of the Melanoma

The data on the characteristics of the pigmentation were collected for all the patients and 220 control subjects. A personal and standardized interview and an examination of the skin on the whole of the body were carried out by a dermatologist. The report of the results used a standard examination report form.

The characteristics of the skin were measured by skin type according to the classification of FITZPATRICK (FITZPATRICK, Arch. Dermatol., Vol. 124, p:869-71, 1988) and evaluated as follows: always burnt never browned (skin type I), always burnt but browned (skin type II); always browned sometimes burnt (skin type III), and always browned never burnt (skin type IV). The color of the eyes was classified as dark (brown or black) or pale (blue, green/hazel or grey) and the original color of the hair (before grey hair) was classified by using 5 categories: red, blond, fair or dark-brown and black. The total count of the naevi of the body (divided into 4 categories: <10, 10-50, 50-100, >100), the presence or the absence of the atypical mole syndrome (SLADE et al., J. Am. Acad. Dermatol., Vol. 32, p: 479-94, 1995) and the presence or absence of solar lentigines were also evaluated by means of a physical examination. Moreover, in the melanoma group, the anatomic localization of the melanomas, the age of the patient upon diagnosis and relevant histopathological data were also reported.

MC1R Genotype, Variable SLC45A2 and OCA2

The polymorphisms of MC1R retained for the genetic analysis were those associated with the red hair color phenotype (RHC alleles) and include c.252C>A p.D84E, c425G>A p.R142H, c.p.R151c, c.476 C>T p.R160W and c.880G>C p.D294H (FLANAGAN et al., Hum. Mol. Genet., Vol. 9, p: 2531-7, 2000; DUFFY et al., Hum. Mol. Genet. Vol. 13, p: 447-61, 2004; REES, Am. J. Hum. Genet. Vol. 75, p: 739-51, 2004). These variants were also associated with the melanoma risk in multiple populations (STURM et al., Pigment Cell Res., Vol. 16, p: 266-72, 2003; REES, Annu. Rev. Genet., Vol. 37, p: 67-90, 2003), and were all identified as generating a loss of function in functional studies (BEAUMONT et al., Hum. Mol. Genet., Vol. 14, p: 2145-54, 2005; BEAUMONT et al., Hum. Mol. Genet., 2007). The two studied variants of SLC45A2 were non-synonymous SNPs (C.1122 C>G, L374F and c.814G>A, E372K) which had been identified beforehand as being associated with normal human pigmentation (GRAF et al., Hum. Mutat. Vol. 25, p: 278-84, 2005). The studied OCA2 variants were the 3 intron SNPs recently presented as being strongly associated with the color of the eyes, of the hair and of the skin pigmentation (rs7495174, rs4778241 and rs4778138) in an Anglo-Celtic population of Queensland. (DUFFY et al., Am. J. Hum. Genet., Vol. 80, p: 241-52, 2007).

All the SNPs were genotyped by using the system for genotyping SNPs by PCR of KBIOSCIENCE. The system for genotyping SNPs by PCR of KBIOSCIENCE is a new homogeneous and fluorescent genotyping system using a single form of allele-specific PCR which is distinct and different from the conventional allele discrimination method. This genotyping method was then validated by an independent genotyping method, the allele discrimination genotyping method by a TAQMAN test of SNPs (APPLIED BIOSYSTEMS). Further, for ⅔ of the SNPs, the verification of the corresponding genotypes was also carried out by sequencing 50-100 DNA samples (ABIPRISM 3130).

The genotyping of MC1R variants was successfully carried out in 828 patients (82%) and 1067 controls (72.78%). The 2 SLC45A2 variants were able to be effectively genotyped in 95% of the patients and of the controls. Genotyping of the OCA2 variants was also able to be carried out for 95% of the patients and of the controls, except for OCA2-rs477824 which was only able to be genotyped on only 81.2% of the patients.

Statistical Analysis

The main element of the statistical analysis was achieved by using the COMPUTER R PACKAGE (version 2.4.1). The significance level for all the tests was set to a level corresponding to an error rate of type I of α=5%. Taking into consideration the number of performed tests and the level of the their association, most of them would have remained significant if a BONFERRONI correction has been applied in order to take the problem of multiple tests into consideration. All the odd reports (ORs) were reported with their confidence level of 95% (CI).

Association of Genetic Factors with Melanoma

The compliance with the HARDY-WEINBERG equilibrium was tested in the controls by using a standard way, one degree of freedom, chi-squared test. The analysis of the individual association of genetic factors with the melanomas (the MC1R, SLC45A2 and OCA2 variables) was carried out by comparing the cases and the controls while using an exact FISHER test of genotypes (coded with 0, 1 and 2, with 0 being the most frequent genotype). The corresponding ORs were set by using a standard regression logical analysis on the data with, for each polymorphism, the reference taken as the most frequent genotype (0). Further, an analysis of the haplotypical diversity was performed on each gene by the expectation maximization algorithm (EM) by using the software package ARLEQUIN (version 3.01) (SCHNEIDER et al., Genetics and Biometry Lab, Dept. of Anthropology, University of Geneva 2000). The linkage disequilibrium (LD) between the pairs of polymorphic sites was measured in the controls by using common statistics D, D′ (LEWONTIN, Genetics, Vol. 49, p: 49-67, 1964) and r (HILL& ROBERTSON, Genetics, Vol. 60, p: 615-28, 1968). Finally, the gene-gene interactions were evaluated by using the same logical regression system.

Association of Clinical Factors with the Melanomas

The individual analyses of usual clinical factors (color of the skin: pale and intermediate versus dark, skin type: I-II versus III-IV, color of the hair: red-blond-pale brown versus dark-brown-black, color of the eyes: light-colored/pale versus black, the number of naevi: <50 versus >50, ephelides and dorsal lentigines: presence versus absence were then also carried out by comparing all the cases and the 220 controls with an exact FISHER genotype test. The corresponding ORs were once again set by using a logical regression analysis on the data.

Association of Genetic Factors with Clinical Factors

Insofar that the three analyzed genes intervene in pigmentation, individual association analyses of the genetic factors for each clinical factor were also carried out, adapted to the case-control status, so that no observed association was due to the association between melanoma and the relevant clinical factors. The corresponding ORs were examined via logical regressions.

Integration of Genetics and Clinical Factors

Because they have been associated with the 3 genes and with the melanoma, clinical factors may create potential confusions when analyzing the association of these genes with the disease. In this part of the analyses, we used a logical model based on a group of clinical and genetic factors in order to check whether the genetic associations with melanoma were still revealed in the presence of clinical factors. Finally, the ORs were recalculated for genetic and clinical factors, finally selected as being the most relevant for the melanoma.

Results

Association of Genetic Factors with the Melanoma

For the controls, the genotype frequencies for all the tested polymorphisms are located in the HARDY-WEINBERG equilibrium.

The results for MC1R are shown in Table I.

TABLE I

| | Cases | Controls | P | OR (CI) |
|---|---|---|---|---|
| MC1R | | | | |
| genotype | | | | |
| 0/0 | 510 | 841 | | reference |
| 1/0 | 280 | 202 | <2.2 × 10⁻¹⁶ | 2.29 (1.85-2.82) |
| 1/1 | 48 | 24 | | 3.3 (2-5.45) |
| RHC alleles | | | | |
| of MC1R | | | | |
| No RHC | 0.793 | 0.887 | | reference |
| alleles | | | | |
| R151C | 0.088 | 0.0042 | | 2.35 (1.78-3.11) |
| R160W | 0.065 | 0.038 | | 1.88 (1.40-2.54) |
| D294H | 0.042 | 0.019 | | 2.49 (1.67-3.71) |
| R142H | 0.013 | 0.008 | | 1.73 (0.92-3.28) |
| D84E | 0.0116 | 0.006 | | 2.10 (1.04-4.25) |

The results have shown that the presence or MC1R RHC variants is strongly associated with the melanoma risk (P<2.20×10⁻¹⁶). The melanoma risk increases with the number of RHC alleles from OR=2.29 with a variant allele, to OR=3.3 with two variant alleles. Consequently, the effect of the polymorphisms on the disease is almost cumulative. The results show the minor allelic frequencies for 5 RHC polymorphisms and, with an exception (R142H), they are all significantly, individually higher in patients with a melanoma than in the controls.

Further, the association of the MC1R variants with the melanoma remains significant after adjustment with the type of skin (P=1.6×10⁻¹⁶), the color of the hair (P=9.3×10⁻⁷), the color of the eyes (P=6.48×10⁻⁷), the color of the hair (P=8×10⁻⁷) and the number of naevi (P=3.3×10⁻⁸).

Tables IIa and IIb summarize the results for the SLC45A2 variants.

TABLE IIa
SLC45A2 genotype for the subjects and controls

| SNPs | Genotype | Cases | Controls | P | OR (CI) |
|---|---|---|---|---|---|
| SLC45A2 | CC | 933 | 1,305 | | reference |
| E272K | CT | 33 | 119 | 1.10 × 10⁻⁶ | 0.39 |
| | | | | | (0.26-0.58) |
| | TT | 2 | 3 | | 0.93 |
| | | | | | (0.16-5.59) |
| MAF | | 0.019 | 0.044 | | |
| SLC45A2 | GG | 895 | 1,160 | | reference |
| L374F | GC | 65 | 246 | 2.12 × 10⁻¹⁵ | 0.34 |
| | | | | | (0.26-0.46) |
| | CC | 5 | 20 | | 0.32 |
| | | | | | (0.24-0.43) |
| MAF | | 0.39 | 0.10 | | |

MAF: minor allelic frequency

TABLE IIb
SLC45A2 genotype for the subjects and controls

| | | Cases | | Controls | | |
|---|---|---|---|---|---|---|
| | | (n = 1882) | | (n = 2794) | | OR (CI) |
| Haplotype | | | | | | |
| SLC45A2 | | | | | | |
| 1 | C₈₁₄G₁₁₂₂ | 1807 | (96) | 2509 | (89.8) | reference |
| 2 | C₈₁₄G₁₁₂₂ | 39 | (2.1) | 164 | (5.9) | 0.33 (0.33-0.47) |
| 3 | T₈₁₄C₁₁₂₂ | 30 | (1.16) | 112 | (4) | 0.37 (0.25-0.56) |
| 4 | T₈₁₄G₁₁₂₂ | 6 | (0.32) | 9 | (0.34) | 0.92 (0.34-2.5) |
| Diplotype | | | | | | |
| SLC45A2 | | | | | | |
| 1/1 | CG/CG | 867 | (92.1) | 1.126 | (80.6) | reference |
| 1/2 | CG/CC | 39 | (4.1) | 148 | (10.6) | 0.34 (0.24-0.49) |
| 1/3 | CG/TC | 28 | (3) | 100 | (7.16) | 0.36 (0.24-0.56) |

The results show that the 2 SLC45A2 variants (L374F and E272K) are closely associated with melanoma (P=2.12×10⁻¹⁵ and 1.10×10⁻⁶ respectively, with an almost complete linkage disequilibrium between both polymorphisms (D=0.036, D′=0.92 and r=0.59). The SLC45A2 374F allelic frequency in the controls is 0.90, while it is 0.96 for the patients (P=1.30 10×10⁻¹¹). Analysis of the haplotype confirmed the association between SLC45A2 and melanoma (the exact FISHER test applied to the haplotype frequencies gives P=3.67×10⁻¹⁵, Table 2b). The haplotype C₈₁₄G₁₁₂₂ is the most frequent, having been found in 96% of the patients and 89.8% of the controls, while the haplotypes C₈₁₄G₁₁₂₂ and T₈₁₄C₁₁₂₂ are significantly more frequent in the controls (a difference which was due to the variant L374F as specified in Table 2b) and therefore have a protective effect against melanoma. A similar analysis of the diplotypes indicates that 2 diplotypes (CG/CC and CG/TC) which are present in up to 17.76% of the controls provides protection against melanoma, while the most common diplotype (CG/CG) is significantly more frequent for patients (92.1%) than for controls (80.1%) (the P value of the exact FISHER test of the diplotypes=1.89×10⁻¹³).

Finally, as the results have shown that the MATP/SLC45A2 gene is strongly related with melanoma, the entire coding sequence (7 exons) of this gene was sequenced for 48 patients affected with melanomas, but no other non-synonymous variant or pathogenic mutation of the SLC45A2 gene was identified.

Interestingly, the association of MATP/SLC45A2 L374F with melanoma remains significant after adaptation with the skin type (P=1.6×10⁻⁶), the color of the hair (P=8.7×10⁻⁷), the color of the eyes (P=1.21×10⁻⁴), and the number of naevi (P=1×10⁻⁵).

In the OCA2 case, a single SNP (OCA2-rs4778138) proved to be slightly associated with the melanoma (P=6.93×10⁻³). The analyses of the haplotypes and diplotypes induced by the 3 SNPs of OCA2 do not indicate any more important significant association of this gene with melanoma, although the TGT haplotype and the diplotype identified earlier as being strongly associated with the color of pale eyes was more frequently observed in patients affected with melanoma than for the controls. However, the association of this SNP with melanoma was not either significant after adaptation with the color of the eyes or after correction for multiple tests.

A logistic model including the genetic factors, having a statistical association with melanoma (Variable MC1R, SLC45A2, L374F, SLC45A2, E272 K and OCA2-rs4778138) was developed. MC1R was taken into account depending on the number of RHC alleles (0, 4 or 2) and the three other ones were coded by combining the less frequent genotypes in order to reduce the genetic model by two parameters without losing any information. The associations of the variants of MC1R and SLC45A2 L374F with melanoma are the only ones which are maintained, suggesting that these genetic factors play a strong and independent role in the disease.

Table III shows the combined calculation of ORs of the MC1R and SLC45A2 L374F genotypes.

TABLE III
Calculation of the combined OR for
the MC1R et SLC45A L374F genotypes

| MC1R | SLC45A2 | | | |
|---|---|---|---|---|
| genotype | genotype | Cases | Controls | OR |
| 0 | 0 | 444 | 561 | Reference |
| 0 | 1 | 32 | 118 | 0.34 (0.23-0.51) |
| 0 | 2 | 3 | 13 | 0.29 (0.09-0.96) |
| 1 | 0 | 249 | 130 | 2.42 (1.89-3.09) |
| 1 | 1 | 20 | 30 | 0.84 (0.47-1.49) |
| 1 | 2 | 2 | 3 | 1.26 (0.22-7.19) |
| 2 | 0 | 39 | 11 | 4.48 (2.29-8.74) |
| 2 | 1 | 3 | 2 | 1.89 (0.38-1.52) |
| 2 | 2 | 0 | 0 | na |

The value 0 designates the most frequent genotypes which are taken as a reference (in the case of MC1R, the absence of RHC variants, in the case of SLC45A2 L374F, the genotype GG). 1 designates the presence of at least one RHC allele for MC1R and of the genotype CG for SLC45A2 L374F, and 2 designates the presence of two RHC variants for MC1R and of the genotype CC for SLC45A2 L374F.

“na” corresponds to “non-applicable”.

Table III shows the respective ORs of all the combinations of genotypes of the two important genetic factors predisposing to melanoma (MC1R and SLC45A2), and illustrates the various risk or protective combinations.

Finally, a study of the potential interactions between the presence of the MC1T variant and of each variant of SLC45A2 or of OCA2 was conducted, but no gene-gene interaction was able to be identified.

Association of Clinical Factors with the Melanoma

The main clinical characteristics of the melanoma cohort and of the 220 controls were studied. This study has shown that the most important risk factors corresponded to a number of naevi>(or equal to) 50 (OR=5.91), to pale eye color (OR=2.55), to skin type I or II (OR=2.35), to fair hair color (OR=2.18) and to solar lentigines (OR=3.19).

Association of Genetic Factors with Clinical Factors

Insofar that these three genes were identified as acting all three on the pigmentation, analyses of individual association of genetic factors on the main factors of clinical risk were also performed and adapted so that the control status may be distinguished from that of the patient affected with melanoma.

The strongest association was that between OCA2 and the color of the eyes (P<2.20×10⁻¹⁶ for each of the three SNPs OCA2), which confirms the recently demonstrated association between these SNPs and the color of the eyes (DUFFY et al., Am. J. Hum. Genet., Vol. 80, p: 241-52, 2007). We also tested the specific association of the TGT haplotype with the color of the eyes and we also find a very significant value of P (P<2; 20×10⁻¹⁶). The three OCA2 variants and the TGT haplotype were also strongly associated with the color of the hair (P<0.0001).

The SLC45A2 L374F SNP proved to be closely associated with the color of the eyes (P=2.09×10⁻⁵), with the color of the hair (P=6.96×10⁻⁸) and with the type of skin (P=6.2×10⁻⁸).

Finally, the MC1R variants have shown significant association with the color of the hair (P=1.49×10⁻⁸) and with the type of skin (P=1.30×10⁻⁸).

To summarize, the type of skin is strongly associated with the SLC45A2 and MC1R variants, the color of the eyes with OCA2 and less significantly with SLC45A2, and the color of the hair with the three genes.

Remarkably, the number of naevi, which was the most important clinical sign of melanoma in our study, was not statistically associated with any one of these pigmentation genes.

Integration of Genetics and Clinical Factors

On the basis of the strongest associations identified in our study among the clinical and genetic factors and the melanoma, we have developed a logical model in order to test the persistence of associations with melanoma and we have recalculated the corresponding ORs when the risk factors were combined.

In this model, only the number of naevi was included since this was the only clinical risk factor which did not show any association with each of the three genes. We have observed that the association of the MC1R and SLC45A2 L374F variants with the melanoma were still highly significant in the presence of a number of naevi (P=1.14×10⁻⁷ and 3.32×10⁻⁶) respectively).

Further, it was easy to calculate the cumulative ORs resulting from the combination of these factors. In this model, the OR of persons having a certain number of naevi>(or equal to) 50, an RHC allele for MC1R and the most frequent genotype SLC45A2 L374F (GG), OR=5.26×3.85=20.25, gives an approximation of the melanoma risk for these persons which is 20 times higher than those having a number of naevi <50, no RHC allele, and one of the protective SLC45A2 genotypes.

Finally, with the present study, it was possible to determine that the genotype GG SLC45A2 L374F is a new significant parameter with which the melanoma risk in a patient may be appreciated, the allele SLC45A2 374F being clearly associated with the melanoma risk, while the allele SLC45A2 374L would itself protect against melanoma.

Additionally, the fact that the effect of this SLC45A2 variant persists after stratification on the pigmentation characteristics or in a logical regression model integrating the 2 genetic risk factors and the number of naevi suggests that as regards the melanoma risk, the information related to the genotype SLC45A2 L374F is not redundant with the pigmentation characteristics and this variant is therefore a strong and independent melanoma risk factor.

The results concerning the marker SLC45A2 L374F were subsequently confirmed on a cohort of Spanish individuals (Fernandez L P et al., Human mutation 0, 1-7, 2008).

On the other hand, the present inventors have also confirmed the results dealing with the markers SLC45A L374F and MC1R on an Italian population.

These results therefore prove without any ambiguity that the markers SLC45A2 L374F and also MC1R RHC are reliable markers of predisposition to melanoma, which may be used alone or in association in order to evaluate very efficiently the risk of predisposition to melanoma in humans in general.

1. An in vitro method intended to identify a subject affected with or having a predisposition to skin cancer, wherein it comprises analyzing a biological sample from said subject by:
a) detection of a polymorphism of a MATP/SLC45A2 gene (SEQ ID NO:1), and/or
b) analysis of the expression of the MATP/SLC45A2 gene.

2. The method according to claim 1, further comprising c) detection of a polymorphism of the MC1R gene (SEQ ID NO:10).

3. The method according to claim 1, wherein said subject is a mammal.

4. The method according to claim 1, wherein said skin cancer is a melanoma.

5. The method according to claim 1, wherein said polymorphism of MATP/SLC45A2 gene associated with a predisposition to skin cancer corresponds to a polymorphism associated with a single nucleotide (SNP).

6. The method according to claim 5, wherein said polymorphism of the MATP/SLC45A2 gene associated with a single nucleotide (SNP) is chosen from the group consisting of the SNP rs16891982 (N nucleotide in position 301 of SEQ ID NO:2, the G nucleotide being associated with skin cancer) and resulting in the presence of a phenylalanine in position 374 of the MATP/SLC45A2 protein (SEQ ID NO:3) and the SNP rs26722 (N nucleotide in position 301 of SEQ ID NO:4, the C nucleotide being associated with skin cancer) resulting in the presence of a glutamate residue in position 272 of the MATP/SLC45A2 protein (SEQ ID NO:3).

7. The method according to claim 5, wherein said polymorphism of the MATP/SLC45A2 gene associated with a single nucleotide (SNP) corresponds to the SNP rs16891982 (N nucleotide in position 301 of SEQ ID NO:2, the G nucleotide being associated with skin cancer) and resulting in the presence of phenylalanine in position 374 of the MATP/SLC45A2 protein (SEQ ID NO:3).

8. The method according to claim 2, wherein said polymorphism of the MC1R gene associated with predisposition to skin cancer corresponds to a polymorphism associated with a single nucleotide (SNP) chosen from the group consisting of the SNP sr1805006 (N nucleotide in position 26 of SEQ ID NO:6, the A nucleotide being associated with a skin cancer) and resulting in the presence of a glutamate in position 84 of the MC1R protein (SEQ ID NO:5), the SNP rs1805007 (N nucleotide in position 301 of SEQ ID NO:7, the T nucleotide being associated with a skin cancer) and resulting in the presence of a cysteine in position 151 of the MC1R protein (SEQ ID NO:5), the SNP rs1805008 (N nucleotide in position 301 of SEQ ID NO:8, the T nucleotide being associated with a skin cancer) and resulting in the presence of a tryptophan in position 160 of the MC1R protein (SEQ ID NO:5), and the SNP rs1805009 (N nucleotide in position 26 of SEQ ID NO:9, the C nucleotide being associated with skin cancer) and resulting in the presence of a histidine in position 294 of the MC1R protein (SEQ ID NO:5).

9. The method according to claim 1, comprising a first step of analyzing the expression of the MATP/SLC45A2 gene in said biological sample, wherein it further comprises comparing the expression level of the MATP/SLC45A2 gene in said biological sample with the expression level of the MATP/SLC45A2 gene in a control sample.

10. A method for treating and/or preventing skin cancer in a patient in need thereof, comprising the administration of an effective amount of a compound which specifically increases the expression of the MATP/SLC45A2 gene in a skin cell.

11. The method according to claim 10, wherein said compound is chosen from the group consisting of the MATP/SLC45A2 protein and its derivatives, a polynucleotide coding for such a protein and a vector comprising such a polynucleotide.

12. A method for selecting in vitro a compound capable of being useful for treating skin cancer, wherein it comprises:
a) obtaining a cell expressing the MATP/SLC45A2 gene;
b) putting said cell in contact with at least one compound to be tested;
c) comparing the expression of the MATP/SLC45A2 gene between steps a) and b); and
d) selecting the compound inducing an increase in the expression level of the MATP/SLC45A2 gene in the cell in step b) relatively to the expression level of said gene in step a).

13. The method according to claim 3, wherein said mammal is a human.
